SEQUENCE LISTING (51 sequences)

SEQ ID NO: 1 - protein, 21 aa, Homo sapiens
MALPVTALLLPLALLLHAARP

SEQ ID NO: 2 - protein, 19 aa, Homo sapiens
MALPVTALLLPLALLLHAA

SEQ ID NO: 3 - protein, 11 aa, Homo sapiens
MALPVTALLLP

SEQ ID NO: 4 - protein, 12 aa, Homo sapiens
PVTALLLPLALL

SEQ ID NO: 5 - protein, 14 aa, Homo sapiens
LLLPLALLLHAARP

SEQ ID NO: 6 - protein, 45 aa, Homo sapiens
TTTPAPRPPTPAPTIASQPLSLRPEACRPAAGGAVHTRGLDFACD

SEQ ID NO: 7 - protein, 27 aa, Homo sapiens
FWVLVVVGGVLACYSLLVTVAFIIFWV

SEQ ID NO: 8 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 9 - protein, 42 aa, Homo sapiens
KRGRKKLLYIFKQPFMRPVQTTQEEDGCSCRFPEEEEGGCEL

SEQ ID NO: 10 - protein, 41 aa, Homo sapiens
RSKRSRLLHSDYMNMTPRRPGPTRKHYQPYAPPRDFAAYRS

SEQ ID NO: 11 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 22 aa, Homo sapiens
GSGATNFSLLKQAGDVEENPGP

SEQ ID NO: 13 - protein, 20 aa, synthetic construct
GGGGSGGGGSGGGGSGGGGS

SEQ ID NO: 14 - protein, 15 aa, synthetic construct
GGGGSGGGGSGGGGS

SEQ ID NO: 15 - protein, 10 aa, synthetic construct
GGGGSGGGGS

SEQ ID NO: 16 - protein, 5 aa, synthetic construct
GGGGS

SEQ ID NO: 17 - protein, 308 aa, Homo sapiens
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 18 - protein, 313 aa, Homo sapiens
VGPFEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSP

SEQ ID NO: 19 - protein, 330 aa, Homo sapiens
GSGATNFSLLKQAGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 20 - protein, 335 aa, Homo sapiens
GSGATNFSLLKQAGDVEENPGPVGPFEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSP

SEQ ID NO: 21 - protein, 710 aa, unidentified
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 22 - protein, 120 aa, Homo sapiens
EVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSS

SEQ ID NO: 23 - protein, 108 aa, Homo sapiens
DIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKR

SEQ ID NO: 24 - protein, 121 aa, Homo sapiens
EVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSS

SEQ ID NO: 25 - protein, 113 aa, Homo sapiens
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIER

SEQ ID NO: 26 - protein, 121 aa, Homo sapiens
GSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 27 - protein, 107 aa, Homo sapiens
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 28 - protein, 122 aa, Homo sapiens
EVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSS

SEQ ID NO: 29 - protein, 107 aa, Homo sapiens
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 30 - protein, 116 aa, Homo sapiens
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVS

SEQ ID NO: 31 - protein, 108 aa, Homo sapiens
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 32 - protein, 119 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 33 - protein, 112 aa, Homo sapiens
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 34 - protein, 122 aa, Homo sapiens
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 35 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 36 - protein, 118 aa, Homo sapiens
EVQLVQSGAEVKKPGASVKVSCKASGYTFTSYYMHWARQAPGQGLEWMGIINPSGGSTSYAQKFQGRVTMTRDTSTSTVYMELSSLRSEDTAVYYCARVVAAAVADYWGQGTLVTVSS

SEQ ID NO: 37 - protein, 112 aa, Homo sapiens
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSLQTPFTFGPGTKVDIK

SEQ ID NO: 38 - protein, 120 aa, Homo sapiens
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 39 - protein, 107 aa, Homo sapiens
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 40 - protein, 120 aa, Homo sapiens
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSG

SEQ ID NO: 41 - protein, 115 aa, Homo sapiens
GSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 42 - protein, 153 aa, Homo sapiens
LKPREVKLVESGGGLVQPGGSLKLSCAASGFDFSRYWMSWVROAPGKGLEWIGEINLDSSTINYTPSLKDKFIISRDNAKNTLYLOMSKVRSEDTALYYCARRYDAMDYWGOGTSVTVSSAKTTAPSVYPLAPVCGDTTGSSVTLGCLVKASQ

SEQ ID NO: 43 - protein, 111 aa, Homo sapiens
ASDIVLTQSPASLAVSLGQRATISCRASESVDDYGISFMNWFOOKPGOPPKLLIYAAPNOGSGVPARFSGSGSGTDFSLNIHPMEEDDTAMYFCQQSKDVRWRHQAGDOTG

SEQ ID NO: 44 - protein, 123 aa, Homo sapiens
SEVQLQQSGAELVKPGASVKMSCKASGYTFTSYNMHVWKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 45 - protein, 123 aa, Homo sapiens; identical to SEQ ID NO: 44

SEQ ID NO: 46 - protein, 111 aa, Homo sapiens
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 47 - protein, 117 aa, Homo sapiens
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 48 - protein, 107 aa, Homo sapiens
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIK

SEQ ID NO: 49 - protein, 118 aa, Homo sapiens
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSS

SEQ ID NO: 50 - protein, 264 aa, synthetic construct
MEFGLSWLFLVAILKGVQCSRDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIKGSTSGSGKPGSGEGSTKGQIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSS

SEQ ID NO: 51 - protein, 264 aa, synthetic construct
MEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSS

(19) United States
(12) Patent Application Publication
(10) Pub. No.: US 2025/0288668 A1
(43) Pub. Date: Sep. 18, 2025

(54) METHODS FOR THE ENHANCEMENT OF THERAPEUTIC EFFECT OF CAR-T CELLS

(71) Applicant: Imago Biosciences, Inc., San Carlos, CA (US)

(72) Inventor: Hugh Y. Rienhoff, Jr., San Carlos, CA (US)

(73) Assignee: Imago Biosciences, Inc., San Carlos, CA (US)

(21) Appl. No.: 18/867,042

(22) PCT Filed: May 19, 2023
(86) PCT No.: PCT/US2023/067222

§ 371 Date: Nov. 19, 2024

Related U.S. Application Data
(60) Provisional application No. 63/365,075, filed on May 20, 2022.

(51) Int. Cl. A61K 40/11; A61K 31/4192; A61K 35/17; A61K 40/15; A61K 40/31

(52) U.S. Cl. CPC A61K 40/11; A61K 31/4192; A61K 35/17; A61K 40/15; A61K 40/31

(57) ABSTRACT

Provided herein is the use of LSD1 inhibitors, in particular bomedemstat, in connection with use and manufacture of immune effector cells such as NK cells and T cells engineered to express a chimeric antigen receptor (CAR), to treat a subject having a disease, associated with expression of a tumor antigen.

This application claims the benefit of priority of U.S. Provisional Application No. 63/365,075, filed May 20, 2022, the disclosure of which is hereby incorporated by reference as if written herein in its entirety.

### INCORPORATION OF SEQUENCE LISTING

The sequence listing that is contained in the file named “IBIS0021-401-PC,” which is 49.7 kilobytes as measured in Microsoft Windows operating system and was created on May 15, 2023, is filed electronically herewith and incorporated herein by reference.

Adoptive transfer of T-lymphocytes (T-cells) engineered to recognize antigens on the surface of tumor cells can be a curative therapy. This is best demonstrated by the effectiveness of a CD-19 targeted chimeric antigen receptor (CAR) T-cell in the treatment of B-cell tumors. Many patients treated in this fashion who achieve a complete remission later relapse. There have been many hypotheses put forth to account for the eventual relapse, one of which is the differentiation and exhaustion of infused T-cells as they repeatedly encounter tumor antigen in the patient characterized as a loss of memory T-cell phenotype.

Fully differentiated T cells are characterized by a distinct epigenetic and gene expression profile compared to naïve or memory T cells. The protein PRDM1 (PR Domain Zinc Finger Protein 1; B lymphocyte-induced maturation protein-1; Blimp-1) is a epigenetic and transcription regulator that has been shown to mediate and is essential for the phenotypic transition from memory to differentiated T cell. A genetic knock-out of PRDM1 in antitumor T cells enhances the durability of antitumor activity in in vitro and in vivo models of adoptive immunotherapy.

However, the implementation of PRDM1 knockout in CAR-T cells necessitates the approval of an entirely new drug product; it is not a solution for effective CAR-T therapies on the market or in current development. Accordingly, a need persists for alternatives to PRDM1-deleted CAR-T cells that can avoid exhaustion through preservation of T cells with a memory phenotype.

Expression of transcription factor (TF) Blimp-1 (PRDM1) is mainly found in activated T cells; this TF is necessary for producing interleukin (IL)-10 by a subset of forkhead box (Fox) p3+ regulatory T cells with an effector phenotype. Blimp-1 coordinates with other TFs to regulate expression of IL-2, IL-21 and IL-10 in effector T lymphocytes. Blimp-1 is also required to induce T cell elimination in the thymus and modulates T cell activation and proliferation in the periphery. Blimp-1 promotes T helper (Th) 2 lineage commitment and limits Th1, Th17 and follicular helper T cell differentiation. In CD8+ T cells, Blimp-1 expression is distinct in heterogeneous populations at the stages of clonal expansion, differentiation, contraction and memory formation when they encounter antigens. Blimp-1 regulates the formation and function of effector and memory CD8+ T cells and their exhaustion.

The Blimp1 (PRDM1) protein recruits to chromatin lysine-specific demethylase 1 (LSD1) which in turn effects epigenetic changes in histone and DNA methylation states to alter the transcriptional profile of lymphocytes. Genetic knock-down of Blimp1 enhances CAR-T target cell killing and inflammatory cytokine secretion together enhancing the clinical efficacy of CAR-T therapy. Accordingly, the inhibition of LSD1 with a small molecule inhibitor provides an alternative to the genetic knock-down of PRDM1.

Furthermore, GFI1 and GFI1b (Growth Factor Independent 1 and 1b) are zinc-finger containing proto-oncogenic transcriptional regulator proteins which are necessary for development and differentiation of erythroid and megakaryocytic lineages, and complex with LSD1 and other proteins to suppress, via histone deacetylase (HDAC) recruitment, a number of genes implicated in multilineage blood cell development and control hematopoietic differentiation. Accordingly, LSD1 inhibitors which inhibit demethylase activity and also inhibit the ability of GFI1 and GFI1b to bind to LSD1 have utility in the immune management of malignancies. LSD1 inhibitors that affect the ability of LSD1 and its complex of proteins and to bind to both the GFI1 and Blimp1 family of transcriptions factors are expected to have advantages over LSD1 inhibitors which bind and inhibit LSD1 alone.

### DETAILED DESCRIPTION

Provided herein is a method of treatment of disease comprising administering:
a therapeutic composition comprising a population of immune effector cells prepared for adoptive transfer, which bind to an antigen on the surface of a target cell; and
a lysine-specific demethylase 1 (LSD1) inhibitor.

Also provided is a method of enhancing the memory phenotype of a population of immune effector cells prepared for adoptive transfer, comprising treating the immune effector cells with a lysine-specific demethylase 1 (LSD1) inhibitor.

Also provided is a method of inhibiting the transition of T cells prepared for adoptive transfer from a memory phenotype to differentiated phenotype, comprising treating the immune effector cells with a lysine-specific demethylase 1 (LSD1) inhibitor.

Also provided is a method of inhibiting or reversing exhaustion in a population of immune effector cells prepared for adoptive transfer, comprising treating the immune effector cells with a lysine-specific demethylase 1 (LSD1) inhibitor.

In some embodiments, the LSD1 inhibitor inhibits both demethylase activity and the ability of Growth Factor Independent 1 and/or Growth Factor Independent 1b (GFI1 and/or GFI1b and Blimp1) to bind to LSD1. In further embodiments, the LSD1 inhibitor is bomedemstat.

In some embodiments, the immune effector cells comprise a chimeric antigen receptor (CAR), e.g., are CAR-T cells.

CARs disclosed herein may have the following further embodiments, which may be present in various combinations to form additional embodiments.

In some embodiments, CARs have a signal/leader peptide that guides the translocation of the extracellular domain of the CAR through the immune effector cell membrane to the cell surface. In some embodiments, the signal/leader peptide is a CD8α signal/leader peptide. In some embodiments, the CD8α signal/leader peptide has an amino acid sequence according to any of SEQ ID NOS: 1-5.

CARs provided herein have an antigen binding domain. In some embodiments, the antigen binding domain comprises a V_{H} chain, a peptide linker and a V_{L} chain. In some embodiments, the V_{H} chain, peptide linker and a V_{L} chain together comprise an antigen binding domain specific for CD2. In some embodiments, the V_{H} chain, peptide linker and a V_{L} chain together comprise an antigen binding domain specific for any of CD3, CD7, FLT3, CS1, and CD33.

In some embodiments, the peptide linker between the V_{H} and V_{L} chains has the amino acid sequence GGGGS₍₁₋₄₎.

In some embodiments, the V_{H} chain and V_{L} chain pair comprise, respectively, amino acid sequences according to any of:
SEQ ID NO:22 and SEQ ID NO:23;
SEQ ID NO:24 and SEQ ID NO:25;
SEQ ID NO:26 and SEQ ID NO:27;
SEQ ID NO:28 and SEQ ID NO:29;
SEQ ID NO:30 and SEQ ID NO:31;
SEQ ID NO:32 and SEQ ID NO:33;
SEQ ID NO:34 and SEQ ID NO:35;
SEQ ID NO:36 and SEQ ID NO:37;
SEQ ID NO:38 and SEQ ID NO:39;
SEQ ID NO:40 and SEQ ID NO:41;
SEQ ID NO:42 and SEQ ID NO:43; and
SEQ ID NO:44 and SEQ ID NO:45;
SEQ ID NO:46 and SEQ ID NO:47; and
SEQ ID NO:48 and SEQ ID NO:49;
or wherein the scFv comprises an amino acid sequence chosen from SEQ ID NO:50 and SEQ ID NO:51.

In some embodiments, the CAR has at least one costimulatory domain selected from CD27, CD28, 4-1BB (CD137), OX40, OX40L, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D, PD-1, ICOS, CDS, ICAM-1, GITR, BAFFR, HVEM (LIGHTR), SLAMF7, NKp80 (KLRF1), CD160, CD19, CD8a, CD8B, IL2R β, IL2R γ, IL7R α, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In some embodiments, the CAR has at least one costimulatory domain selected from CD2, CD8a, CD8b CD28, ICOS, CD27, OX40, 4-1BB, NKG2D and CD4. In some embodiments, the CAR has at least one costimulatory domain selected from CD28 and 4-1BB. In some embodiments, at least one costimulatory domain is 4-1BB. In some embodiments, the 4-1BB domain has the amino acid sequence according to SEQ ID NO: 9. In some embodiments, at least one costimulatory domain is CD28. In some embodiments, the CD28 domain has the amino acid sequence according to SEQ ID NO:10. In some embodiments, the CAR has one co-stimulatory domain. In some embodiments, the CAR has two co-stimulatory domains. In some embodiments, the CAR has a plurality of co-stimulatory domains.

In some embodiments, the signaling domain is a CD3 zeta (CD3ζ) domain. In some embodiments, the CD3ζ domain has the amino acid sequence according to SEQ ID NO: 11

Also provided herein are chimeric antigen receptors (CARs; and transgenic T-cell receptors, TCRs) comprising polypeptides as disclosed herein, e.g., as disclosed in Tables 2, 3, 12 and 13, and immune effector cells expressing them. A CAR is a recombinant fusion protein comprising: 1) an extracellular ligand-binding domain, i.e., an antigen-recognition domain, 2) a hinge domain, 3) a transmembrane domain, and 4) a cytoplasmic signaling domain, 5) and optionally, a co-stimulatory domain.

Methods for CAR design, delivery and expression, and the manufacturing of clinical-grade CAR-T cell populations are known in the art. CAR designs are generally tailored to each cell type.

The extracellular ligand-binding domain of a chimeric antigen receptor recognizes and specifically binds an antigen, typically a surface-expressed antigen of a malignant cell. The extracellular ligand-binding domain specifically binds an antigen when, for example, it binds the antigen with an affinity constant or affinity of interaction (K_{D}) between about 0.1 μM to about 10 μM, or about 0.1 μM to about 1 μM, or about 0.1 μM to about 100 nM. Methods for determining the affinity of interaction are known in the art. An extracellular ligand-binding domain can also be said to specifically bind a first polymorphic variant of an antigen when it binds it selectively over a second polymorphic variant of the same antigen.

An extracellular ligand-binding domain suitable for use in a CAR may be any antigen-binding polypeptide, a wide variety of which are known in the art. In some instances, the extracellular ligand-binding domain is a single chain Fv (scFv). Other antibody-based recognition domains (cAb VHH (camelid antibody variable domains) and humanized versions thereof, 1gNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use. In some embodiments, the extracellular ligand-binding domain is constructed from a natural binding partner, or a functional fragment thereof, to a target antigen. For example, CARs in general may be constructed with a portion of the APRIL protein, targeting the ligand for the B-Cell Maturation Antigen (BCMA) and Transmembrane Activator and CAML Interactor (TACI), effectively co-targeting both BCMA and TACI for the treatment of multiple myeloma.

The targeted antigen to which the CAR binds via its extracellular ligand-binding domain (or antigen-binding domain) may be an antigen that is expressed on a malignant myeloid (AML) cell, T cell or other cell. Antigens expressed on malignant myeloid (AML) cells include CD33, FLT3, CD123, and CLL-1. Antigens expressed on T cells include CD2, CD3, CD4, CD5, CD7, TCRα (TRAC), TCRβ, CD70 and CD1a. Antigens expressed on malignant plasma cells include BCMA, CS1, CD38, CD79A, CD79B, CD138, and CD19. Antigens expressed on malignant B cells include CD19, CD20, CD21, CD22, CD23, CD24, CD25, CD27, CD38, and CD45.

Typically, the extracellular ligand-binding domain is linked to the intracellular domain of the chimeric antigen receptor by a transmembrane (TM) domain. A peptide hinge connects the extracellular ligand-binding domain to the transmembrane domain. The hinge is generally any oligo- or polypeptide that functions to link the transmembrane domain to the extracellular ligand-binding domain. In particular, the hinge is used to provide more flexibility and accessibility for the extracellular antigen binding domain. A hinge may comprise up to 300 amino acids, or 10 to 100 amino acids, or 25 to 50 amino acids. The hinge may be derived from all or parts of naturally-occurring molecules such as CD28, 4-1BB (CD137), OX-40 (CD134), CD3ζ, the T cell receptor α or β chain, CD45, CD4, CD5, CD8, CD8a, CD9, CD16, CD22, CD33, CD37, CD64, CD80, CD86, ICOS, CD154 or from all or parts of an antibody constant region. Alternatively, the hinge may be a synthetic sequence that corresponds to a naturally occurring hinge sequence or the hinge region may be an entirely synthetic hinge sequence. In some embodiments, the hinge domain comprises a part of human CD8α, FcγRIIIa receptor, or IgG1, and have at least 80%, 90%, 95%, 97%, or 99% sequence identity thereto. In some embodiments, the CD8& hinge comprises the amino acid sequence according to SEQ ID NO:6.

The transmembrane domain traverses the cell membrane, anchors the CAR to the T cell surface, and connects the extracellular ligand-binding to the intracellular (cytoplasmic) signaling domain, thus impacting expression of the CAR on the T cell surface.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. In some embodiments, the transmembrane domain is a CD28 transmembrane domain comprising the amino acid sequence according to SEQ ID NO:7. Alternatively, the transmembrane domain can be synthetic and comprise predominantly hydrophobic amino acid residues (e.g., leucine and valine). In some cases, a triplet of phenylalanine, tryptophan and valine will be found at each end of a synthetic transmembrane domain. In some embodiments, the transmembrane domain is derived from the T-cell surface glycoprotein CD8 alpha chain isoform 1 precursor (NP_001139345.1) or CD28. A short oligo- or polypeptide linker, such as between 2 and 10 amino acids in length, may form the linkage between the transmembrane domain and the endoplasmic domain of the CAR. In some embodiments, the CAR has more than one transmembrane domain, which can be a repeat of the same transmembrane domain, or can be different transmembrane domains.

After antigen recognition, the cytoplasmic signaling domain transmits a signal to the immune effector cell, activating at least one of the normal effector functions of the immune effector cell. Effector function of a T cell, for example, may be cytolytic activity or helper activity including the secretion of cytokines. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (FcγRIIA), DAP10, DAP12, CD79a, CD79b, FcγRIγ, FcγRIIIγ, FcεRIβ (FCERIB), and FcεRIγ (FCERIG).

First-generation CARs typically have the cytoplasmic signaling domain from the CD3 chain, which is the primary transmitter of signals from endogenous TCRs. Second-generation CARs add cytoplasmic signaling domains from various co-stimulatory protein receptors (e.g., CD28, 4-1BB, ICOS) to the cytoplasmic signaling domain of the CAR to provide additional signals to the T cell.

A costimulatory domain is derived from the intracellular signaling domains of costimulatory proteins that enhance cytokine production, proliferation, cytotoxicity, and/or persistence in vivo. Preclinical studies have indicated that the second generation of CAR designs improves the antitumor activity of T cells. More recent, third-generation, and later generation, CARs combine multiple costimulatory domains to further augment potency. T cells grafted with these CARs have demonstrated improved expansion, activation, persistence, and tumor-eradicating efficiency independent of costimulatory receptor/ligand interaction.

For example, the cytoplasmic signaling domain of the CAR can be designed to comprise the signaling domain (e.g., CD3ζ), either by itself or combined with any other desired cytoplasmic domain(s) useful in the context of the CAR of the invention. For example, the cytoplasmic domain of the CAR can comprise a signaling domain (e.g., CD3ζ) chain portion and a costimulatory signaling region. The co-stimulatory signaling region refers to a portion of the CAR comprising the intracellular domain of a co-stimulatory molecule. Examples of such molecules include CD27, CD28, 4-1BB (CD137), OX40, OX40L, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D, PD-1, ICOS, CDS, ICAM-1, GITR, BAFFR, HVEM (LIGHTR), SLAMF7, NKp80 (KLRF1), CD160, CD19, CD8a, CD8B, IL2R β, IL2R γ, IL7R α, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the cytoplasmic signaling domain is a CD3 zeta (CD3ζ) signaling domain. In some embodiments, the co-stimulatory domain comprises the cytoplasmic domain of CD28, 4-1BB, or a combination thereof. In some cases, the co-stimulatory signaling region contains 1, 2, 3, or 4 cytoplasmic domains of one or more intracellular signaling and/or co-stimulatory molecules.

In some embodiments, CAR expression vector also includes a selection marker, for example CD34 or truncated CD34, and a P2A sequence. The CD34 or TrCD34 is translated as a separate protein from the CAR and allows selection for cells containing the CAR through anti-CD34 antibodies or other binding agents attached to magnetic beads or a solid surface. In some embodiments, the nucleic acid encoding the CAR may optionally encode a peptide or polypeptide linker and a CD34 domain or a truncated CD34 (TrCD34) domain. This peptide or polypeptide linker and a CD34 domain or truncated CD34 (TrCD34) domain is translated as a separate protein and is, therefore, not part of the CAR polypeptide. The separately translated CD34 domain or TrCD34 domain can be used for selection of CAR-bearing cells, e.g., by using an anti-CD34 antibody or other CD34 binding agent attached to a surface or to magnetic beads. In some embodiments, the CD34 domain has the amino acid sequence according to SEQ ID NO:17. In some embodiments, the TrCD34 domain has the amino acid sequence according to SEQ ID NO:18. In some embodiments, the polypeptide linker is P2A. In some embodiments, the P2A linker has the amino acid sequence according to SEQ ID NO:12. In some embodiments, the P2A linker and the CD34 domain are contiguous and have the amino acid sequence according to SEQ ID NO: 19. In some embodiments, the P2A linker and the TrCD34 domain are contiguous and have the amino acid sequence according to SEQ ID NO: 20.

### ENUMERATED EMBODIMENTS

Accordingly, although other embodiments may be found throughout the disclosure, provided herein are the following embodiments:

Embodiment 1. A method of treatment of disease comprising administering: a therapeutic composition comprising a population of immune effector cells prepared for adoptive transfer, which bind to an antigen on the surface of a target cell; and a lysine-specific demethylase 1 (LSD1) inhibitor.

Embodiment 2. The any of method of Embodiment 1, wherein the LSD1 inhibitor is administered concurrently with the therapeutic composition comprising a population of immune effector cells prepared for adoptive transfer.

Embodiment 3. The method of Embodiment 1, wherein the LSD1 inhibitor is administered after the therapeutic composition comprising a population of immune effector cells prepared for adoptive transfer.

Embodiment 4. The method of Embodiment 1, wherein the LSD1 inhibitor is administered before the therapeutic composition comprising a population of immune effector cells prepared for adoptive transfer.

Embodiment 5. The method of Embodiment 1, wherein the LSD1 inhibitor is administered after adoptively transferred immune cell exhaustion is observed.

Embodiment 6. The method of any of Embodiments 1-5, wherein the disease is cancer, and the target cell is a cancer cell.

Embodiment 7. A method of enhancing the memory phenotype of a population of immune effector cells prepared for adoptive transfer, comprising treating the immune effector cells with a lysine-specific demethylase 1 (LSD1) inhibitor.

Embodiment 8. A method of inhibiting the transition of T cells prepared for adoptive transfer from a memory phenotype to differentiated phenotype, comprising treating the immune effector cells with a lysine-specific demethylase 1 (LSD1) inhibitor.

Embodiment 9. A method of inhibiting or reversing exhaustion in a population of immune effector cells prepared for adoptive transfer, comprising treating the immune effector cells with a lysine-specific demethylase 1 (LSD1) inhibitor.

Embodiment 10. The method of any of Embodiments 7-9, wherein the LSD1 inhibitor is administered after adoptively transferred immune cell exhaustion is observed.

Embodiment 11. The method of any of Embodiments 1-10, wherein the LSD1 inhibitor inhibits both demethylase activity and the ability of Growth Factor Independent 1 and/or Growth Factor Independent 1b (GFI1 and/or GFI1b and Blimp!) to bind to LSD1.

Embodiment 12. The method of any of Embodiments 1-1, wherein the LSD1 inhibitor is a compound of any of Formulas I-VI as disclosed herein.

Embodiment 13. The method of Embodiment 12, wherein the LSD1 inhibitor is bomedemstat.

Embodiment 14. The method of Embodiment 13, wherein the bomedemstat is administered in an amount titrated to a platelet count of 50-150 k/uL once daily.

Embodiment 15. The method of Embodiment 12, wherein the LSD1 inhibitor is chosen from bomedemstat (IMG-7289), iadademstat (ORY-1001), vafidemstat (ORY-2001), pulrodemstat (CC-90011), seclidemstat (SP-2577), INCB059872, TAS1440, SYHA1807, RO7051790, GSK2879552, MC2580, and DDP-38003.

Embodiment 16. The method of Embodiment 15, wherein the LSD1 inhibitor is chosen from bomedemstat (IMG-7289), iadademstat (ORY-1001), and pulrodemstat (CC-90011).

Embodiment 17. The method of any of Embodiments 1-16, wherein the immune effector cells are chosen from T cells and natural killer cells.

Embodiment 18. The method of Embodiment 17, wherein the immune effector cells express at least one chimeric antigen receptor (CAR).

Embodiment 19. The method of Embodiment 18, wherein the immune effector cells are NK cells.

Embodiment 20. The method of Embodiment 18, wherein the immune effector cells are T cells.

Embodiment 21. The method of Embodiment 19 wherein the immune effector cell is a CAR-T cell.

Embodiment 22. The method of Embodiment 18 or 20 wherein the CAR comprises:
an optional CD8a leader polypeptide;
an antigen binding domain;
a hinge;
a transmembrane domain;
at least one co-stimulatory domain; and
a signaling domain.

Embodiment 23. The method of Embodiment 18 or 20 wherein the CAR comprises:
an extracellular domain comprising an optional CD8a leader polypeptide, an antigen binding domain, and a hinge;
a transmembrane domain; and
an intracellular domain comprising at least one co-stimulatory domain, and a signaling domain.

Embodiment 24. The method of either of Embodiments 22 or 23, wherein said transmembrane domain is chosen from a CD28 transmembrane domain and a CD8 transmembrane domain.

Embodiment 25. The method of either of Embodiments 22 or 23, wherein said intracellular domain is chosen from a CD28 intracellular signaling domain, an OX40L intracellular signaling domain, and a CD3-zeta (CD3ζ) signaling domain.

Embodiment 26. The method of any of Embodiments 18-23, wherein the CAR comprises the elements from a proximal N-terminus to a distal C-terminus.

Embodiment 27. The method of any of Embodiments 18-23, wherein the CAR(s) specifically binds at least one antigen expressed on a malignant T cell.

Embodiment 28. The method of Embodiment 27, wherein the antigen expressed on the malignant T cell is selected from one or more of CD2, CD3, CD4, CD5, CD7, TRAC, CD70, CD1a, and TCRβ.

Embodiment 29. The method of any of Embodiments 18-28, wherein the CAR(s) specifically binds at least one antigen expressed on a malignant plasma cell.

Embodiment 30. The method of Embodiment 29, wherein the antigen expressed on the malignant plasma cell is selected from one or more of BCMA, CS1, CD38, CD79A, CD79B, CD138, and CD19.

Embodiment 31. The method of any of Embodiments 18-28, wherein the CAR(s) specifically binds at least one antigen expressed on a malignant B cell.

Embodiment 32. The method of Embodiment 31, wherein the antigen expressed on a malignant B cell is selected from one or more of CD19, CD20, CD21, CD22, CD23, CD24, CD25, CD27, CD38, and CD45.

Embodiment 33. The method of Embodiment 32, wherein the antigen expressed on a malignant B cell is selected from one or more of CD19 and CD20.

Embodiment 34. The method of Embodiment 33, wherein the antigen expressed on a malignant B cell is CD19.

Embodiment 35. The method of any of Embodiments 1-34, wherein the antigen binding domain comprises a V_{H} chain, a peptide linker and a V_{L} chain (an scFv).

Embodiment 36. The method of Embodiment 35, wherein the scFv comprises a V_{H} chain and a V_{L} chain pair comprising, respectively, amino acid sequences according to any of:
SEQ ID NO:22 and SEQ ID NO:23;
SEQ ID NO:24 and SEQ ID NO:25;
SEQ ID NO:26 and SEQ ID NO:27;
SEQ ID NO:28 and SEQ ID NO:29;
SEQ ID NO:30 and SEQ ID NO:31;
SEQ ID NO:32 and SEQ ID NO:33;
SEQ ID NO:34 and SEQ ID NO:35;
SEQ ID NO:36 and SEQ ID NO:37;
SEQ ID NO:38 and SEQ ID NO:39;
SEQ ID NO:40 and SEQ ID NO:41;
SEQ ID NO:42 and SEQ ID NO:43; and
SEQ ID NO:44 and SEQ ID NO:45;
SEQ ID NO:46 and SEQ ID NO:47; and
SEQ ID NO:48 and SEQ ID NO:49;
or wherein the scFv comprises an amino acid sequence chosen from SEQ ID NO:50 and SEQ ID NO:51.

Embodiment 37. The method of Embodiment 35 or 36, wherein the peptide linker between the V_{H} and V_{L} chains has the amino acid sequence GGGGS₍₁₋₄₎.

Embodiment 38. The method of any of Embodiments 1-37, wherein the CAR has a co-stimulatory domain.

Embodiment 39. The method of Embodiment 38 wherein the CAR has a plurality of co-stimulatory domains.

Embodiment 40. The method of Embodiment 39, wherein the CAR has two co-stimulatory domains.

Embodiment 41. The method of Embodiment 38, wherein the co-stimulatory domain(s) is/are chosen from 4-1BB, CD28, OX-40 and NKG2D.

Embodiment 42. The method of any of Embodiments 1-41, wherein the CAR-T cell is deficient in a subunit of the T cell receptor complex and/or is deficient in at least one or more antigens to which the one or more CARs specifically binds.

Embodiment 43. The method of Embodiment 42, wherein the subunit of the T cell receptor complex is selected from one or more of TCRα, TCRβ, TCRδ, TCRγ, CD3ε, CD3γ, CD3δ, and an extracellular domain of CD3ζ.

Embodiment 44. The method of any of Embodiments 1-43, wherein the method is performed in a human subject with cancer.

Embodiment 45. The method of any of Embodiments 1-44, wherein the cancer is a hematologic malignancy.

Embodiment 46. The method of Embodiment 45, wherein the hematologic malignancy is a myeloproliferative neoplasm.

Embodiment 47. The method as recited in Embodiment 46, wherein the myeloproliferative neoplasm is myelofibrosis selected from primary myelofibrosis (PMF) and post PV/ET myelofibrosis (MF).

Embodiment 48. The method as recited in Embodiment 47, wherein the myeloproliferative neoplasm is post PV/ET myelofibrosis (MF).

Embodiment 49. The method as recited in Embodiment 46, wherein the myeloproliferative neoplasm is polycythemia vera.

Embodiment 50. The method as recited in Embodiment 46, wherein the myeloproliferative neoplasm is essential thrombocythemia.

Embodiment 51. The method of Embodiment 45, wherein the hematologic malignancy is acute myeloid leukemia (AML).

Embodiment 52. The method of Embodiment 45, wherein the hematologic malignancy is chronic myeloid leukemia (CML).

Embodiment 53. The method of Embodiment 45, wherein the hematologic malignancy is multiple myeloma.

Embodiment 54. The method of Embodiment 45, wherein the hematologic malignancy is a T-cell malignancy.

Embodiment 55. The method of Embodiment 54, wherein the T cell malignancy is selected from T-cell acute lymphoblastic leukemia/lymphoma (T-ALL), human T-cell leukemia virus type 1-positive (HTLV-1+) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotrophic intestinal T-cell lymphoma, Mycosis fungoides, Nodal peripheral T-cell lymphoma with TFH phenotype, Peripheral T-cell lymphoma (PTCL), NOS, Primary cutaneous γδ T-cell lymphoma, Primary cutaneous CD8+ aggressive epidermotropic cytotoxic T-cell lymphoma, Primary cutaneous acral CD8+ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

Embodiment 56. The method of Embodiment 55, wherein the T cell malignancy is T-cell acute lymphoblastic leukemia (T-ALL).

Embodiment 57. The method of Embodiment 54, wherein the T cell malignancy is non-Hodgkin's lymphoma.

Embodiment 58. The method of Embodiment 54, wherein the T cell malignancy is T-cell chronic lymphocytic leukemia (T-CLL).

Embodiment 59. The method of Embodiment 45, wherein the hematologic malignancy is a B-cell malignancy.

Embodiment 60. The method of Embodiment 59, wherein the B-cell malignancy is selected from diffuse large B cell lymphoma (DLBCL), chronic lymphocytic leukemia (CLL)/small lymphocytic lymphoma (SLL), and B cell-precursor acute lymphoblastic leukemia (ALL).

Embodiment 61. The method of Embodiment 45, wherein the hematologic malignancy is a plasma cell malignancy.

Embodiment 62. The method of Embodiment 61, wherein the plasma cell malignancy is selected from lymphoplasmacytic lymphoma, plasmacytoma and multiple myeloma.

Also provided are corresponding uses of the recited elements in each of the foregoing embodiments—e.g., immune effector cells and lysine-specific demethylase 1 (LSD1) inhibitors—in the treatment of cancer, and in the manufacture of a medicament for the treatment of cancer, and in kits comprising instructions for the treatment of cancer.

### CAR Design and Construction

The chimeric antigen receptor (CAR) construct, which encodes the chimeric receptor can be prepared in conventional ways. Since, for the most part, natural sequences are employed, the natural genes are isolated and manipulated, as appropriate (e.g., when employing a Type II receptor, the immune signaling receptor component may have to be inverted), so as to allow for the proper joining of the various components. Thus, the nucleic acid sequences encoding for the N-terminal and C-terminal proteins of the chimeric receptor can be isolated by employing the polymerase chain reaction (PCR), using appropriate primers which result in deletion of the undesired portions of the gene. Alternatively, restriction digests of cloned genes can be used to generate the chimeric construct. In either case, the sequences can be selected to provide for restriction sites which are blunt-ended, or have complementary overlaps.

The various manipulations for preparing the chimeric construct can be carried out in vitro and in particular embodiments the chimeric construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like.

It is contemplated that the chimeric construct can be introduced into immune effector cells as naked DNA or in a suitable vector. Methods of stably transfecting immune effector cells by electroporation using naked DNA are known in the art. See, e.g., U.S. Pat. No. 6,410,319. Naked DNA generally refers to the DNA encoding a chimeric receptor of the present invention contained in a plasmid expression vector in proper orientation for expression. Advantageously, the use of naked DNA reduces the time required to produce immune effector cells expressing the chimeric receptor of the present invention.

Alternatively, a viral vector (e.g., a retroviral vector, adenoviral vector, adeno-associated viral vector, or lentiviral vector) can be used to introduce the chimeric construct into immune cell, e.g., T cells. Suitable vectors for use in accordance with the method of the present invention are non-replicating in the immune effector cells of the subject. A large number of vectors are known which are based on viruses, where the copy number of the virus maintained in the cell is low enough to maintain the viability of the cell. Illustrative vectors include the pFB-neo vectors (STRATAGENE™) as well as vectors based on HIV, SV40, EBV, HSV or BPV. Once it is established that the transfected or transduced immune effector cell is capable of expressing the chimeric receptor as a surface membrane protein with the desired regulation and at a desired level, it can be determined whether the chimeric receptor is functional in the host cell to provide for the desired signal induction (e.g., production of Rantes, Mip1-alpha, GM-CSF upon stimulation with the appropriate ligand).

Engineered CARs may be introduced into CAR-bearing immune effector cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type Ill systems using a suitable Cas protein such Cas3, Cas4, Cas5, Cas5e (or CasD), Cash, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) may also be used. See, e.g., Shearer RF and Saunders DN, “Experimental design for stable genetic manipulation in mammalian cell lines: lentivirus and alternatives,” Genes Cells 2015 January; 20 (1): 1-10.

Editing strategies, such as CRISPR guided base editors (CBE), enable efficient genome editing with minimal DNA scission. The application for CBE in the context of engineered T cells includes silencing of TCR and HLA expression and, to prevent fratricide, target antigen expression. Editing is achieved by coupling inactivated Cas9 with nucleotide deaminase, guided by a target specific RNA sequence to convert C to T. Conversion efficiency can be enhanced by the inclusion of uracil DNA-glycosylase inhibitors. CBE can mediate gene silencing through the introduction of premature stop codons or splice site disruption resulting on non-sense mediated decay (NMD) of mRNA transcripts. Multiple iterations of CBE are known in the art. Common base editors include, without limitation, BE3, VQR-BE3, EQR-BE3, VRER-BE3, SaBE3, SaKKH-BE3, SaBE3-Gam, TAM, CRISPR X, ABE, and YEE-BE3.

Amino acid sequences for selected components which may be used to construct a CAR are disclosed below in Table 1 and Table 2, below. As will be appreciated by those of skill in the art, in some embodiments, the sequences below may be varied by elongation, truncation, and substitution of amino acids. Additionally, in some embodiments, alternate variants are not listed here (e.g., additional P2A sequences and their function in inducing polypeptide “cleavage” by ribosomal skipping are known in the art).

TABLE 1
Amino acid sequences of different CAR components.

| | SEQ ID | |
|---|---|---|
| Functional domains | NO: | Amino acid sequence |
| CD8α signal/leader | SEQ ID | MALPVTALLLPLALLLHAARP |
| peptide (variant 1) | NO: 1 | |
| CD8α signal/leader | SEQ ID | MALPVTALLLPLALLLHAA |
| peptide (variant 2) | NO: 2 | |
| CD8α signal/leader | SEQ ID | MALPVTALLLP |
| peptide (variant 3) | NO: 3 | |
| CD8α signal/leader | SEQ ID | PVTALLLPLALL |
| peptide (variant 4) | NO: 4 | |
| CD8α signal/leader | SEQ ID | LLLPLALLLHAARP |
| peptide (variant 5) | NO: 5 | |
| CD8α hinge | SEQ ID | TTTPAPRPPTPAPTIASQPLSLRPEACRPAAGGAVHTR |
| | NO: 6 | GLDFACD |
| CD28 Transmembrane | SEQ ID | FWVLVVVGGVLACYSLLVTVAFIIFWV |
| (Tₘ) domain | NO: 7 | |
| Surface glycoprotein | SEQ ID | MALPVTALLLPLALLLHAARPSQFRVSPLDRTWNLG |
| CD8 alpha chain | NO: 8 | ETVELKCQVLLSNPTSGCSWLFQPRGAAASPTFLLYL |
| isoform 1 precursor | | SQNKPKAAEGLDTQRFSGKRLGDTFVLTLSDFRREN |
| (NP_001139345.1) | | EGYYFCSALSNSIMYFSHFVPVFLPAKPTTTPAPRPPT |
| | | PAPTIASQPLSLRPEACRPAAGGAVHTRGLDFACDIY |
| | | IWAPLAGTCGVLLLSLVITLYCNHRNRRRVCKCPRP |
| | | VVKSGDKPSLSARYV |
| 4-1BB costimulatory | SEQ ID | KRGRKKLLYIFKQPFMRPVQTTQEEDGCSCRFPEEEE |
| domain | NO: 9 | GGCEL |
| CD28 costimulatory | SEQ ID | RSKRSRLLHSDYMNMTPRRPGPTRKHYQPYAPPRDF |
| domain | NO: 10 | AAYRS |
| CD3 zeta (ζ) | SEQ ID | RVKFSRSADAPAYKQGQNQLYNELNLGRREEYDVL |
| | NO: 11 | DKRRGRDPEMGGKPRRKNPQEGLYNELQKDKMAE |
| | | AYSEIGMKGERRRGKGHDGLYQGLSTATKDTYDAL |
| | | HMQALPPR |
| P2A peptide | SEQ ID | GSGATNFSLLKQAGDVEENPGP |
| | NO: 12 | |
| (GGGGS)₄ linker | SEQ ID | GGGGSGGGGSGGGGSGGGGS |
| | NO: 13 | |
| (GGGGS)₃ linker | SEQ ID | GGGGSGGGGSGGGGS |
| | NO: 14 | |
| (GGGGS)₂ linker | SEQ ID | GGGGSGGGGS |
| | NO: 15 | |
| (GGGGS)₁ linker | SEQ ID | GGGGS |
| | NO: 16 | |
| hCD34 | SEQ ID | MPRGWTALCLLSLLPSGFMSLDNNGTATPELPTQGT |
| | NO: 17 | FSNVSTNVSYQETTTPSTLGSTSLHPVSQHGNEATTN |
| | | ITETTVKFTSTSVITSVYGNTNSSVQSQTSVISTVFTTP |
| | | ANVSTPETTLKPSLSPGNVSDLSTTSTSLATSPTKPYT |
| | | SSSPILSDIKAEIKCSGIREVKLTQGICLEQNKTSSCAE |
| | | FKKDRGEGLARVLCGEEQADADAGAQVCSLLLAQS |
| | | EVRPQCLLLVLANRTEISSKLQLMKKHQSDLKKLGIL |
| | | DFTEQDVASHQSYSQKTLIALVTSGALLAVLGITGYF |
| | | LMNRRSWSPI |
| TrhCD34 | SEQ ID | VGPFEAMPRGWTALCLLSLLPSGFMSLDNNGTATPE |
| | NO. 18 | LPTQGTFSNVSTNVSYQETTTPSTLGSTSLHPVSQHG |
| | | NEATTNITETTVKFTSTSVITSVYGNTNSSVQSQTSVI |
| | | STVFTTPANVSTPETTLKPSLSPGNVSDLSTTSTSLAT |
| | | SPTKPYTSSSPILSDIKAEIKCSGIREVKLTQGICLEQN |
| | | KTSSCAEFKKDRGEGLARVLCGEEQADADAGAQVC |
| | | SLLLAQSEVRPQCLLLVLANRTEISSKLQLMKKHQS |
| | | DLKKLGILDFTEQDVASHQSYSQKTLIALVTSGALLA |
| | | VLGITGYFLMNRRSWSP |
| P2A-hCD34 | SEQ ID | GSGATNFSLLKQAGDVEENPGPMPRGWTALCLLSLL |
| | NO: 19 | PSGFMSLDNNGTATPELPTQGTFSNVSTNVSYQETTT |
| | | PSTLGSTSLHPVSQHGNEATTNITETTVKFTSTSVITS |
| | | VYGNTNSSVQSQTSVISTVFTTPANVSTPETTLKPSLS |
| | | PGNVSDLSTTSTSLATSPTKPYTSSSPILSDIKAEIKCS |
| | | GIREVKLTQGICLEQNKTSSCAEFKKDRGEGLARVL |
| | | CGEEQADADAGAQVCSLLLAQSEVRPQCLLLVLAN |
| | | RTEISSKLQLMKKHQSDLKKLGILDFTEQDVASHQS |
| | | YSQKTLIALVTSGALLAVLGITGYFLMNRRSWSPI |
| P2A-TrhCD34 | SEQ ID | GSGATNFSLLKQAGDVEENPGPVGPFEAMPRGWTA |
| | NO: 20 | LCLLSLLPSGFMSLDNNGTATPELPTQGTFSNVSTNV |
| | | SYQETTTPSTLGSTSLHPVSQHGNEATTNITETTVKFT |
| | | STSVITSVYGNTNSSVQSQTSVISTVFTTPANVSTPET |
| | | TLKPSLSPGNVSDLSTTSTSLATSPTKPYTSSSPILSDI |
| | | KAEIKCSGIREVKLTQGICLEQNKTSSCAEFKKDRGE |
| | | GLARVLCGEEQADADAGAQVCSLLLAQSEVRPQCL |
| | | LLVLANRTEISSKLQLMKKHQSDLKKLGILDFTEQD |
| | | VASHQSYSQKTLIALVTSGALLAVLGITGYFLMNRR |
| | | SWSP |
| Human-Herpes | SEQ ID | MPRGWTALCLLSLLPSGFMSLDNNGTATPELPTQGT |
| Simplex Virus-1 (HSV) - | NO: 21 | FSNVSTNVSYQETTTPSTLGSTSLHPVSQHGNEATTN |
| thymidine kinase | | ITETTVKFTSTSVITSVYGNTNSSVQSQTSVISTVFTTP |
| (TK) | | ANVSTPETTLKPSLSPGNVSDLSTTSTSLATSPTKPYT |
| | | SSSPILSDIKAEIKCSGIREVKLTQGICLEQNKTSSCAE |
| | | FKKDRGEGLARVLCGEEQADADAGAQVCSLLLAQS |
| | | EVRPQCLLLVLANRTEISSKLQLMKKHQSDLKKLGIL |
| | | DFTEQDVASHQSYSQKTLIALVTSGALLAVLGITGYF |
| | | LMNRRSWSPTGEGGGGGDLGGVKLPHLFGKRLVEA |
| | | RMASYPCHQHASAFDQAARSRGHSNRRTALRPRRQ |
| | | QEATEVRLEQKMPTLLRVYIDGPHGMGKTTTTQLLV |
| | | ALGSRDDIVYVPEPMTYWQVLGASETIANIYTTQHR |
| | | LDQGEISAGDAAVVMTSAQITMGMPYAVTDAVLAP |
| | | HVGGEAGSSHAPPPALTLLLDRHPIAVMLCYPAARY |
| | | LMGSMTPQAVLAFVALIPPTLPGTNIVLGALPEDRHI |
| | | DRLAKRQRPGERLDLAMLAAIRRVYGLLANTVRYL |
| | | QGGGSWWEDWGQLSGTAVPPQGAEPQSNAGPRPHI |
| | | GDTLFTLFRAPELLAPNGDLYNVFAWALDVLAKRL |
| | | RPMHVFILDYDQSPAGCRDALLQLTSGMVQTHVTTP |
| | | GSIPTICDLARTFAREMGEAN |

### Cell-Specific Variations

The CAR components and construction methods disclosed above are suitable for use in T cells and other immune effector cells, but are not exhaustive. Certain variations may be useful in subsets of cells, and are known in the art.

For example, in NK cells, the TM domain may be chosen or adapted from NKG2D, FcγRIIIa, NKp44, NKp30, NKp46, actKIR, NKG2C, or CD8α, the α, β or ζ chain of the T-cell receptor, CD28, CD27, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA1 (CD11a, CD18), ICOS (CD278), 4-1BB (CD137), GITR, CD40, BAFFR, HVEM (LIGHTR), SLAMF7, NKp80 (KLRF1), CD160, CD19, IL2R β, IL2R γ, IL7R α, ITGA1, VLA1, CD49a, ITGA4, IA4, CD49D, ITGA6, VLA6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTBA, Ly108), SLAM (SLAMF1, CD150, IPO3), BLAME (SLAMF8), SELPLG (CD162), LTBR, or PAG/Cbp. NK cells also express a number of transmembrane adapters that are triggered via association with activating receptors, providing an NK cell specific signal enhancement. For example, the TM adapter can be chosen or adapted from FceR1γ (ITAMx1), CD3ζ (ITAMx3), DAP12 (ITAMx1), or DAP10 (YxxM/YINM). In some embodiments, the TM domains and adapters may be paired, e.g.: NKG2D and DAP10, FcγRIIIa and CD3ζ or FceR1γ, NKp44 and DAP12, NKp30 and CD3ζ or FceR1γ, NKp46 and CD3ζ or FceR1γ, actKIR and DAP12, and NKG2C and DAP12.

In some embodiments, in NK cells, the hinge domain may be chosen or adapted from, e.g., NKG2, TMα, or CD8.

In some embodiments, in NK cells, the intracellular signaling and/or costimulatory domain may comprise one or more of: CD137/41BB (TRAF, NFkB), DNAM-1 (Y-motif), NKp80 (Y-motif), 2B4 (SLAMF)::ITSM, CRACC (CS1/SLAMF7)::ITSM, CD2 (Y-motifs, MAPK/Erk), CD27 (TRAF, NFkB), PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, or TGF beta; one or more integrins (e.g., multiple integrins); a cytokine receptor associated with persistence, survival, or metabolism, such as IL-2/15R::Jak1/3, STAT3/5, PI3K/mTOR, and MAPK/ERK; a cytokine receptor associated with activation, such as IL-18R::NFkB. a cytokine receptor associated with IFN-γ production, such as IL-12R::STAT4; a cytokine receptor associated with cytotoxicity or persistence, such as IL-21R::Jak3/Tyk2, or STAT3; and a TM adapter, as disclosed above. In some embodiments, the NK cell CAR comprises three signaling domains, a TM domain, and optionally, a TM adapter.

Table 2 below discloses the sequences of examples of V_{H} and V_{L} domains which target the recited antigens, or the scFvs themselves, which contain the V_{H} and V_{L} domains. These sequences may be incorporated into CARs along with elements from Table 1 or as disclosed herein. Additional V_{H} and V_{L} domains and scFvs for a variety or targets are known in the art.

TABLE 2
Amino acid sequences of selected scFvs or their variable heavy (V_{H}) and
variable light (V_{L}) chains.

| | SEQ ID | |
|---|---|---|
| ScFv sequences | NO: | Amino acid sequence |
| CD2 heavy chain | SEQ ID | EVKLEESGAELVKPGASVKLSCRTSGFNIKDTYIH |
| variable region (1) | NO: 22 | WVKQRPEQGLKWIGRIDPANGNTKYDPKFQDKAT |
| | | VTADTSSNTAYLQLSSLTSEDTAVYYCVTYAYDG |
| | | NWYFDVWGAGTAVTVSS |
| CD2 light chain | SEQ ID | DIKMTQSPSSMYVSLGERVTITCKASQDINSFLSWF |
| variable region (1) | NO: 23 | QQKPGKSPKTLIYRANRLVDGVPSRFSGSGSGQDY |
| | | SLTISSLEYEDMEIYYCLQYDEFPYTFGGGTKLEM |
| | | KR |
| CD2 heavy chain | SEQ ID | EVQLEESGAELVRPGTSVKLSCKASGYTFTSYWM |
| variable region (2) | NO: 24 | HWIKQRPEQGLEWIGRIDPYDSETHYNEKFKDKAI |
| | | LSVDKSSSTAYIQLSSLTSDDSAVYYCSRRDAKYD |
| | | GYALDYWGQGTSVTVSS |
| CD2 light chain | SEQ ID | DIVMTQAAPSVPVTPGESVSISCRSSKTLLHSNGNT |
| variable region (2) | NO: 25 | YLYWFLQRPGQSPQVLIYRMSNLASGVPNRFSGSG |
| | | SETTFTLRISRVEAEDVGIYYCMQHLEYPYTFGGG |
| | | TKLEIER |
| CD3 heavy chain | SEQ ID | GSQVQLQQSGAELARPGASVKMSCKASGYTFTRY |
| variable region (OKT | NO: 26 | TMHWVKQRPGQGLEWIGYINPSRGYTNYNQKFK |
| 3) | | DKATLTTDKSSSTAYMQLSSLTSEDSAVYYCARY |
| | | YDDHYCLDYWGQGTTLTVSS |
| CD3 light chain | SEQ ID | QIVLTQSPAIMSASPGEKVTMTCSASSSVSYMNWY |
| variable region (OKT | NO: 27 | QQKSGTSPKRWIYDTSKLASGVPAHFRGSGSGTSY |
| 3) | | SLTISGMEAEDAATYYCQQWSSNPFTFGSGTKLEI |
| | | NR |
| CD3 heavy chain | SEQ ID | EVQLVESGGGLVQPGGSLRLSCAASGYSFTGYTM |
| variable region | NO: 28 | NWVRQAPGKCLEWVALINPYKGVSTYNQKFKDR |
| (UCHT1) | | FTISVDKSKNTAYLQMNSLRAEDTAVYYCARSGY |
| | | YGDSDWYFDVWGQGTLVTVSS |
| CD3 heavy chain | SEQ ID | DIQMTQSPSSLSASVGDRVTITCRASQDIRNYLNW |
| variable region | NO: 29 | YQQKPGKAPKLLIYYTSRLESGVPSRFSGSGSGTD |
| (UCHT1) | | YTLTISSLQPEDFATYYCQQGNTLPWTFGCGTKVEI |
| | | K |
| CD7 heavy chain | SEQ ID | EVQLVESGGGLVKPGGSLKLSCAASGLTFSSYAMS |
| variable region | NO: 30 | WVRQTPEKRLEWVASISSGGFTYYPDSVKGRFTIS |
| | | RDNARNILYLQMSSLRSEDTAMYYCARDEVRGYL |
| | | DVWGAGTTVTVS |
| CD7 light chain | SEQ ID | DIQMTQTTSSLSASLGDRVTISCSASQGISNYLNWY |
| variable region | NO: 31 | QQKPDGTVKLLIYYTSSLHSGVPSRFSGSGSGTDYS |
| | | LTISNLEPEDIATYYCQQYSKLPYTFGGGTKLEIKR |
| FLT3 heavy chain | SEQ ID | EVQLVQSGAEVKKPGASVKVSCKASGYTFTSYYM |
| variable region | NO: 32 | HWVRQAPGQGLEWMGIINPSGGSTSYAQKFQGRV |
| | | TMTRDTSTSTVYMELSSLRSEDTAVYYCARGVGA |
| | | HDAFDIWGQGTTVTVSS |
| FLT3 light chain | SEQ ID | DVVMTQSPLSLPVTPGEPASISCRSSQSLLHSNGNN |
| variable region | NO: 33 | YLDWYLQKPGQSPQLLIYLGSNRASGVPDRFSGSG |
| | | SDTDFTLQISRVEAEDVGVYYCMQGTHPAISFGQG |
| | | TRLEIK |
| FLT3 heavy chain | SEQ ID | EVQLVQSGAEVKKPGSSVKVSCKASGGTFSSYAIS |
| variable region | NO: 34 | WVRQAPGQGLEWMGGIIPIFGTANYAQKFQGRVTI |
| | | TADKSTSTAYMELSSLRSEDTAVYYCATFALFGFR |
| | | EQAFDIWGQGTTVTVSS |
| FLT3 light chain | SEQ ID | DIQMTQSPSSLSASVGDRVTITCRASQSISSYLNWY |
| variable region | NO: 35 | QQKPGKAPKLLIYAASSLQSGVPSRFSGSGSGTDFT |
| | | LTISSLQPEDLATYYCQQSYSTPFTFGPGTKVDIK |
| FLT3 heavy chain | SEQ ID | EVQLVQSGAEVKKPGASVKVSCKASGYTFTSYYM |
| variable region | NO: 36 | HWARQAPGQGLEWMGIINPSGGSTSYAQKFQGRV |
| | | TMTRDTSTSTVYMELSSLRSEDTAVYYCARVVAA |
| | | AVADYWGQGTLVTVSS |
| FLT3 light chain | SEQ ID | DVVMTQSPLSLPVTPGEPASISCRSSQSLLHSNGYN |
| variable region | NO: 37 | YLDWYLQKPGQSPQLLIYLGSNRASGVPDRFSGSG |
| | | SGTDFTLKISRVEAEDVGVYYCMQSLQTPFTFGPG |
| | | TKVDIK |
| CS1 heavy chain | SEQ ID | QVQLQQPGAELVRPGASVKLSCKASGYSFTTYWM |
| variable region | NO: 38 | NWVKQRPGQGLEWIGMIHPSDSETRLNQKFKDKA |
| | | TLTVDKSSSTAYMQLSSPTSEDSAVYYCARSTMIA |
| | | TRAMDYWGQGTSVTVSS |
| CS1 light chain | SEQ ID | DIVMTQSQKSMSTSVGDRVSITCKASQDVITGVAW |
| variable region | NO: 39 | YQQKPGQSPKLLIYSASYRYTGVPDRFTGSGSGTD |
| | | FTFTISNVQAEDLAVYYCQQHYSTPLTFGAGTKLE |
| | | LK |
| CD33 heavy chain | SEQ ID | QVQLQQPGAEVVKPGASVKMSCKASGYTFTSYYI |
| variable region | NO: 40 | HWIKQTPGQGLEWVGVIYPGNDDISYNQKFQGKA |
| | | TLTADKSSTTAYMQLSSLTSEDSAVYYCAREVRLR |
| | | YFDVWGQGTTVTVSSSG |
| CD33 light chain | SEQ ID | GSEIVLTQSPGSLAVSPGERVTMSCKSSQSVFFSSS |
| variable region | NO: 41 | QKNYLAWYQQIPGQSPRLLIYWASTRESGVPDRFT |
| | | GSGSGTDFTLTISSVQPEDLAIYYCHQYLSSRTFGQ |
| | | GTKLEIKR |
| CD19 heavy chain | SEQ ID | LKPREVKLVESGGGLVQPGGSLKLSCAASGFD |
| variable region | NO: 42 | FSRYWMSWVROAPGKGLEWIGEINLDSSTINY |
| | | TPSLKDKFIISRDNAKNTLYLOMSKVRSEDTAL |
| | | YYCARRYDAMDYWGOGTSVTVSSAKTTAPS |
| | | VYPLAPVCGDTTGSSVTLGCLVKASQ |
| CD19 light chain | SEQ ID | ASDIVLTQSPASLAVSLGQRATISCRASESVDD |
| variable region | NO: 43 | YGISFMNWFOOQKPGOPPKLLIYAAPNOGSGVP |
| | | ARFSGSGSGTDFSLNIHPMEEDDTAMYFCQQS |
| | | KDVRWRHQAGDOTG |
| CD20 heavy chain | SEQ ID | SEVQLQQSGAELVKPGASVKMSCKASGYTFT |
| variable region | NO: 44 | SYNMHVWKQTPGQGLEWIGAIYPGNGDTSYN |
| | | QKFKGKATLTADKSSSTAYMQLSSLTSEDSAD |
| | | YYCARSNYYGSSYWFFDVWGAGTTVTVSS |
| CD20 light chain | SEQ ID | SEVQLQQSGAELVKPGASVKMSCKASGYTFT |
| variable region | NO: 45 | SYNMHVWKQTPGQGLEWIGAIYPGNGDTSYN |
| | | QKFKGKATLTADKSSSTAYMQLSSLTSEDSAD |
| | | YYCARSNYYGSSYWFFDVWGAGTTVTVSS |
| BCMA light chain | SEQ ID | DIVLTQSPPSLAMSLGKRATISCRASESVTILGS |
| | NO: 46 | HLIHWYQQKPGQPPTLLIQLASNVQTGVPARF |
| | | SGSGSRTDFTLTIDPVEEDDVAVYYCLQSRTIP |
| | | RTFGGGTKLEIK |
| BCMA heavy chain | SEQ ID | QIQLVQSGPELKKPGETVKISCKASGYTFTDYS |
| | NO: 47 | INWVKRAPGKGLKWMGWINTETREPAYAYD |
| | | FRGRFAFSLETSASTAYLQINNLKYEDTATYFC |
| | | ALDYSYAMDYWGQGTSVTVSS |
| HER2 light chain | SEQ ID | DIQMTQSPSSLSASVGDRVTITCKASQDINSYL |
| | NO: 48 | SWFQQKPGKAPKTLIYRANRLVDGVPSRFSGS |
| | | GSGQDYTLTISSLQPEDFATYYCLQYDEFPWT |
| | | FGQGTKVEIK |
| HER2 heavy chain | SEQ ID | QVQLVQSGSELKKPGASVKVSCKASGYTFTN |
| | NO: 49 | YGVNWVRQAPGQGLEWMGWINTHTGEPTYA |
| | | EEFKGRFVFSLDTSVSTAYLQISSLKAEDTAVY |
| | | YCARDDYYVRVDYWGQGTTVTVSS |
| CD5 scFv (1) | SEQ ID | MEFGLSWLFLVAILKGVQCSRDIQMTQSPSSM |
| | NO: 50 | SASLGDRVTITCRASQDINSYLSWFQQKPGKSP |
| | | KTLIYRANRLVDGVPSRFSGSGSGTDYTLTISS |
| | | LQYEDFGIYYCQQYDESPWTFGGGTKLEIKGS |
| | | TSGSGKPGSGEGSTKGQIQLVQSGPGLKKPGG |
| | | SVRISCAASGYTFTNYGMNWVKQAPGKGLR |
| | | WMGWINTHTGEPTYADDFKGRFTFSLDTSKST |
| | | AYLQINSLRAEDTATYFCTRRGYDWYFDVWG |
| | | QGTTVTVSS |
| CD5 scFv (2) | SEQ ID | MEFGLSWLFLVAILKGVQCSRDIKMTQSPSSM |
| | NO: 51 | YASLGERVTITCKASQDINSYLSWFQQKPGKS |
| | | PKTLIYRANRLVDGVPSRFSGSGSGQDYSLTIS |
| | | SLDYEDMGIYYCQQYDESPWTFGGGTKLEIKG |
| | | STSGSGKPGSGEGSTKGQIQLVQSGPELKKPGE |
| | | TVKISCKASGYTFTNYGMNWVKQAPGKGLR |
| | | WMGWINTHTGEPTYADDFKGRFAFSLETSAST |
| | | AYLQINNLKNEDTATYFCTRRGYDWYFDVW |
| | | GAGTTVTVSS |

Additional cancer antigens and scFvs which bind thereto are known in the art and may be implemented in the CARs, CAR-expressing immune effector cells, and methods comprising them disclosed herein.

CARs to be constructed and expressed in immune effector cells according to methods known in the art and disclosed herein may be of the form:
   |-[(optional leader)(V_{L})(linker)(V_{H})(hinge)(TMD)(costim)ₙ(effector)]-|,
   wherein the leader sequence, VL, linker, hinge, transmembrane domain, costimulatory domain, optional second and optional third costimulatory domains (i.e., n is 1, 2, or 3), effector domain and optional P2a sequence and tag are disclosed above in Tables 1 and 2, or otherwise known in the art. Where an example includes P2A-(Tr) CD34, this reflects the fact that the DNA sequences encoding these are present in the nucleic acid sequences encoding the CARs. However, when the mRNA encoding the CAR is translated, the CAR portion distal to the P2A-TrCD34 and the P2A-TrCD34 (or P2A-CD34) are translated as separate polypeptides. Thus, the amino acid sequence of a CAR does not include P2A-(Tr) CD34.

Similar CAR variations are possible as well. For example, CARs may have the form:
   |-[(optional leader)(V_{H})(linker)(V_{L})(hinge)(TMD)(costim)ₙ(effector)]-|,
   wherein the sequences are disclosed above in Tables 1 and 2. Accordingly, also provided are CARs which are identical to those above, except that the positions in the sequence of the V_{H} and V_{L} are reversed.

Also provided herein are immune effector cells bearing one or more CARs according to the first, second, and/or third aspect. In some embodiments, the CAR-bearing immune effector cell is a chimeric antigen receptor T-cell (CAR-T cell) or a chimeric antigen receptor natural killer cell (CAR-NK cell).

In some embodiments, the CAR-bearing immune effector cell has additional features that reduce or eliminate fratricide, alloreactivity and/or graft-versus-host reactions. In some embodiments, the CAR-bearing immune effector cell further comprises a suicide gene. In some embodiments, CAR-bearing immune effector cell is deficient in at least one or more antigens to which the one or more CARs specifically binds. In some embodiments where the CAR-bearing immune effector cell is a CAR-T cell, T cell receptor mediated signaling is blocked in the CAR-T cell. In some embodiments, the CAR-T cell is deficient in a subunit of the T cell receptor complex. In some embodiments, the subunit of the T cell receptor complex is selected from one or more of TCRα, TCRβ, TCRδ, TCRγ, CD3ε, CD3γ, CD3δ, and an extracellular domain of CD3ζ.

Some of these features of CAR-bearing immune effector cells may be introduced through CRISPR/Cas9-mediated editing to delete the gene that encodes the protein for which deficiency is desired. For example, if the CAR-bearing immune effector cell targets CD2, it may be desirable to delete endogenous CD2 and TRAC from the CAR-bearing immune effector cell to reduce or prevent fratricide. The deletion of the CD2 and TRAC genes, in this case, may be confirmed by PCR on the gene-edited cell, sometimes called deep sequencing. Examples of guide RNAs used for gene editing and primers to confirm such editing are known in the art.

### Immune Effector Cells

In some embodiments, the CAR-bearing immune effector cell specifically binds at least one cancer-related antigen expressed on a cancer cell. In some embodiments, the cancer cell is a malignant T cell. In some embodiments, the antigen expressed on the malignant T cell is selected from one or more of CD2, CD3, CD4, CD5, CD7, TCRα (TRAC), and TCRβ. In some embodiments, the cancer cell is a malignant plasma cell. In some embodiments, the antigen expressed on the malignant plasma cell is selected from one or more of BCMA, CS1, CD38, CD79A, CD79B, CD138, and CD19. In some embodiments, the cancer cell is a malignant B cell. In some embodiments, the antigen expressed on a malignant B cell is selected from one or more of CD19, CD20, CD21, CD22, CD23, CD24, CD25, CD27, CD38, and CD45. the antigen expressed on a malignant B cell is selected from one or more of CD19 and CD20.

Immune effector cells as disclosed herein may include T cells, NK cells, and others, for example macrophages, and subtypes thereof.

Any of these immune effector cells may be transduced with a CAR using techniques known in the art. The resulting CAR-bearing immune effector cells may be used in the immunotherapy of disease, for example cancer, by adoptive cell transfer (ACT) into a subject in need. CAR-bearing immune effector cells include CAR-T cells, CAR-NK cells and CAR-macrophages.

Immune effector cells for use in ACT may be autologous or allogeneic. In some embodiments, the use of allogeneic cells permits deliberate polymorphic mismatch between donor and recipient, which offers certain advantages discussed below.

### T Cells

T cells are immune cells which express a T cell receptor (TCR) on their surface. Effector T cells include cytotoxic (CD8+) T cells, helper (CD4+) T cells, viral-specific cytotoxic T cells, memory T cells, gamma delta (γδ) T cells.

T cells may be primary T cells, or may be derived from progenitor cells. T cells can be derived from various sources, including peripheral or cord blood cells, stem cells, or induced pluripotent stem cells (iPSCs), Methods of enriching/isolating, differentiating, and otherwise producing T cells are known in the art.

### NK Cells

Natural killer (NK) cells are traditionally considered innate immune effector lymphocytes which mediate host defense against pathogens and antitumor immune responses by targeting and eliminating abnormal or stressed cells through a variety of receptors on their surfaces, as well as through antibody-dependent cellular cytotoxicity (ADCC), via through the integration of signals from activating and inhibitory receptors.

Natural killer (NK) cells are an alternative to T cells for allogeneic cellular immunotherapy since they have been administered safely without major toxicity, do not cause graft versus host disease (GvHD) or require HLA matching or gene editing, naturally recognize and eliminate malignant cells, and are amendable to cellular engineering. This means that allogeneic CAR-NK cells can be manufactured in bulk, cryopreserved, and shipped to patients on demand, without the need for a patient-specific engineering process that can take time and be expensive. Additionally, NK cells have the potential to activate other components of the immune system and induce long-term anticancer immune memory.

Autologous CAR-T cell therapy, on the other hand, requires engineering the patient's own cells, which can be time-consuming and may not be possible for some patients who do not have enough suitable T cells. Even when autologous CAR-T cells are successfully manufactured and administered, patients typically only receive a single dose due to the risk of acute side effects like ICANS and CRS. This limits the potential for repeat dosing and tailored dosing based on individual patient response. The cost of autologous CAR-T therapy can also be prohibitive for many patients.

In contrast, allogeneic NK cell therapy offers several advantages, including the potential for repeat dosing, response-based dosing, and tailored dosing based on individual patient response. NK cells have also been well tolerated without the risk of graft-versus-host disease, neurotoxicity, or cytokine release syndrome associated with other cell-based therapies. Umbilical cord blood units with preferred characteristics for enhanced clinical activity can be selected as a source for NK cells. Furthermore, NK cells have the ability to bridge innate immunity and induce a multi-clonal adaptive immune response that can lead to long-term anticancer immune memory.

NK cells may be primary NK cells, or may be derived from progenitor cells. NK cells can be derived from various sources, including peripheral or cord blood cells, stem cells, or induced pluripotent stem cells (iPSCs), Methods of enriching/isolating, differentiating, and otherwise producing NK cells are known in the art.

NK cells express a number of transmembrane (TM) adapters that signal activation, that are triggered via association with activating receptors. This provides an NK cell specific signal enhancement via engineering the TM domains from activating receptors, and thereby harness endogenous adapters. The TM adapter can be any endogenous TM adapter capable of signaling activation. In some embodiments, the TM adapter may be chosen from FceR1γ (ITAMx1), CD3ζ (ITAMx3), DAP12 (ITAMx1), or DAP10 (YxxM/YINM), NKG2D, FcγRIIIa, NKp44, NKp30, NKp46, actKIR, NKG2C, CD8a, and IL15Rb.

### LSD1 Inhibitors

Many LSD1 inhibitors are known in the art. Examples of LSD1-inhibiting compounds which may be used in the methods disclosed herein include those shown and referenced below. The LSD1 inhibitors of Formulas I-VI below, including bomedemstat, inhibit both demethylase activity and the ability of GFI1 and GFI1b and Blimp1 to bind to LSD1.

In some embodiments, the LSD1 inhibitor is a compound of Formula I:

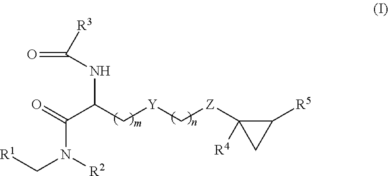

or a salt thereof, wherein:
Y is chosen from a bond, NR^{4a}, O, C(O)NH, NHC(O), S, SO₂, and CH₂;
Z is chosen from a bond, NR^{4b}, O, C(O)NH, NHC(O), S, SO₂, and CH₂;
m is an integer from 0 to 5;
n is an integer from 0 to 3;
R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;
R⁴, R^{4a}, and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;
optionally with the proviso that when Y═CH₂, R⁴═H, and Z═R^{4b}, then m+n≠3.

In some embodiments, the LSD1 inhibitor is a compound of Formula II:

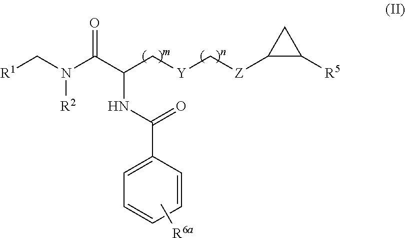

or a salt, polymorph, or solvate thereof, wherein:
Y is chosen from a bond, NR^{4a}, O, C(O)NH, NHC(O), S, SO₂, CHOH, and CH₂;
Z is chosen from a bond, NR^{4b}, O, C(O)NH, NHC(O), S, SO₂, and CH₂;
m is chosen from 0, 1, 2, 3, 4, and 5;
n is chosen from 0, 1, 2, and 3;
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4a} and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
R^{6a} is chosen from heteroaryl, cyano, and S(O)₂N(CH₃)₂;
each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;
optionally with the proviso that when Y═CH₂ and Z═R^{4h}, then m+n≠3.

In some embodiments, the LSD1 inhibitor has Formula IIIa or IIIb:

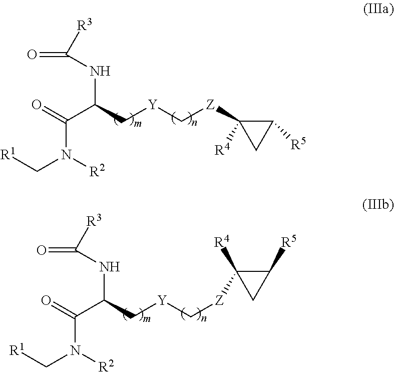

or a salt thereof, wherein:
Y is chosen from a bond, NR^{4a}, O, C(O)NH, NHC(O), S, SO₂, and CH₂;
Z is chosen from a bond, NR^{4b}, O, C(O)NH, NHC(O), S, SO₂, and CH₂;
m is an integer from 0 to 5;
n is an integer from 0 to 3;
R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;
R⁴, R^{4a}, and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;
with the proviso that when Y═CH₂ and Z═R^{4h}, then m+n≠3.

In some embodiments, Z is NR^{4b}.

In some embodiments, R^{4b} is chosen from methyl and hydrogen.

In some embodiments, the alkyl, whether by itself or as a named part of another non-cyclic substituent, is C₁-C₈ alkyl.

In some embodiments, R³ is chosen from aryl, arylalkyl, heteroaryl, and heteroarylalkyl, any of which may be optionally substituted with between 0 and 3 R⁶ groups.

In some embodiments, R³ is chosen from aryl and heteroaryl, any of which may be optionally substituted with between 0 and 3 R⁶ groups.

In some embodiments, m is an integer from 0 to 1; Y is chosen from NR^{4a}, O, S, SO₂, and CH₂; n is an integer from 1 to 3; and R^{4a} is chosen from hydrogen and alkyl.

In some embodiments, m is 0; Y is CH₂; and n is an integer from 1 to 2.

In some embodiments, n is 1. In some embodiments, n is 2. In some embodiments, n is 3.

In some embodiments, R³ is 5-6 membered monocyclic or 8-12 membered bicyclic heteroaryl, in which between one and five ring members may be heteroatoms chosen from N, O, and S, and which may be optionally substituted with between 0 and 3 R⁶ groups.

In some embodiments, R³ is 5-6 membered monocyclic heteroaryl, in which between one and four ring members may be heteroatoms chosen from N, O, and S, and which may be optionally substituted with between 0 and 3 R⁶ groups.

In some embodiments, each R⁶ is chosen from lower alkyl, halogen, lower alkoxy, OCF₃ and CF₃.

In some embodiments, R³ is chosen from

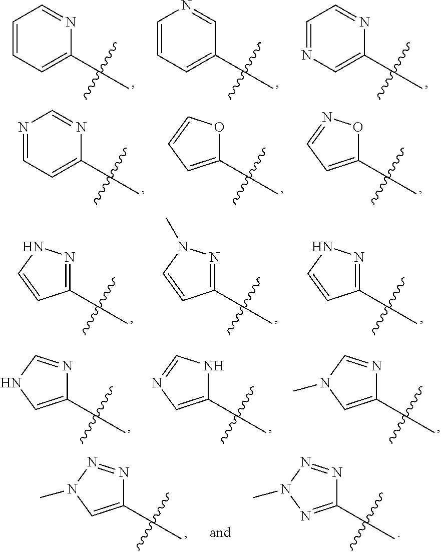

In some embodiments, R⁴ is hydrogen.

In some embodiments, R⁴ is methyl.

In some embodiments, the nitrogen-containing heterocycloalkyl or heteroaryl ring formed by R¹ and R² together with the nitrogen to which they are attached contains 3 to eight atoms.

In some embodiments, R¹ and R² are taken together to form a nitrogen-containing heterocycloalkyl, which may be optionally substituted with between 0 and 3 R⁶ groups.

In some embodiments, the nitrogen-containing heterocycloalkyl formed by R¹ and R² together with the nitrogen to which they are attached is chosen from:

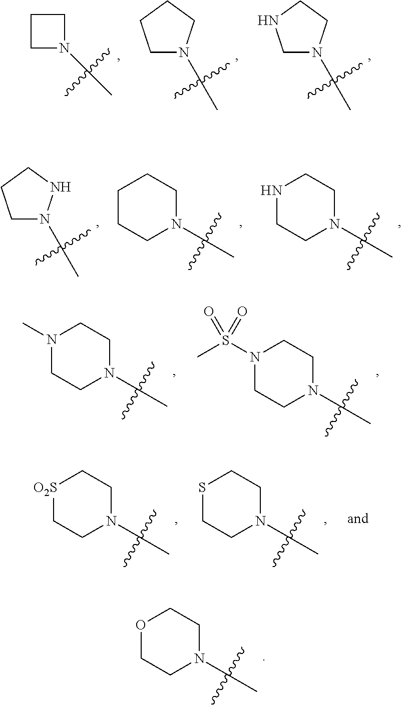

In some embodiments, the nitrogen-containing heterocycloalkyl formed by R¹ and R² together with the nitrogen to which they are attached is chosen from:

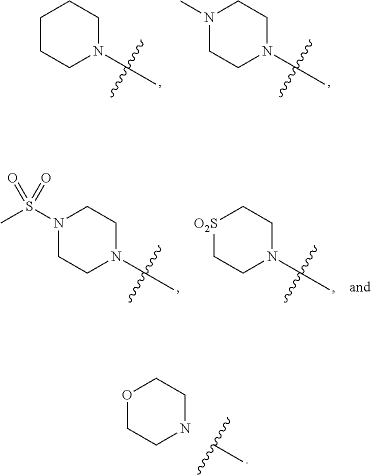

In some embodiments, the nitrogen-containing heterocycloalkyl formed by R¹ and R² together with the nitrogen to which they are attached is

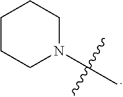 [SMILES: *N1CCCCC1]

In some embodiments, the nitrogen-containing heterocycloalkyl formed by R¹ and R² together with the nitrogen to which they are attached is

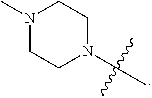 [SMILES: *N1CCN(C)CC1]

In some embodiments, the nitrogen-containing heterocycloalkyl formed by R¹ and R² together with the nitrogen to which they are attached is

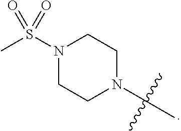 [SMILES: *N1CCN(S(C)(=O)=O)CC1]

In some embodiments, the nitrogen-containing heterocycloalkyl formed by R¹ and R² together with the nitrogen to which they are attached is

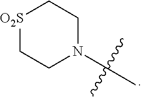 [SMILES: *N1CCS(=O)(=O)CC1]

In some embodiments, the nitrogen-containing heterocycloalkyl formed by R¹ and R² together with the nitrogen to which they are attached is

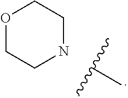

In some embodiments, n is 2 or 3.

In some embodiments, R¹ and R² are taken together with the nitrogen to which they are attached form a nitrogen-containing heteroaryl, which may be optionally substituted with between 0 and 3 R⁶ groups.

In some embodiments, the nitrogen-containing heteroaryl is chosen from pyrrole, imidazole, and pyrazole.

In some embodiments, R⁵ is aryl, which may be optionally substituted with between 0 and 3 R⁶ groups.

In some embodiments, R⁵ is phenyl, which may be optionally substituted with between 0 and 3 R⁶ groups.

In some embodiments, n is 2 or 3.

In some embodiments, R⁵ is heteroaryl, which may be optionally substituted with between 0 and 3 R⁶ groups.

In some embodiments, R⁵ is a 5-6 membered monocyclic or 8-12 membered bicyclic heteroaryl, in which between one and five ring members may be heteroatoms chosen from N, O, and S, and which may be optionally substituted with between 0 and 3 R⁶ groups.

In some embodiments, R⁵ is a 5-6 membered monocyclic heteroaryl, in which between one and five ring members may be heteroatoms chosen from N, O, and S, and which may be optionally substituted with 1 or 2 R⁶ groups.

In some embodiments, R⁵ is chosen from:

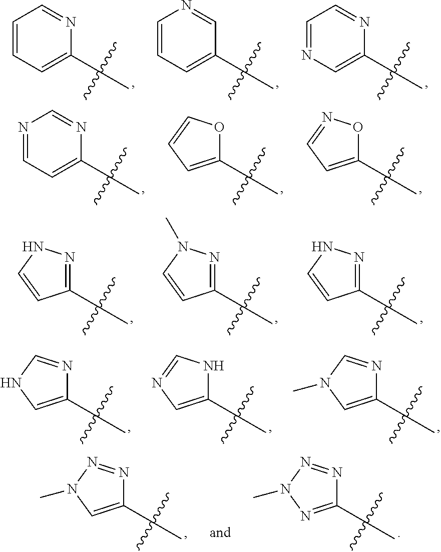

In some embodiments, n is 2 or 3.

In some embodiments, R³ is aryl, optionally substituted with between 0 and 3 R⁶ groups.

In some embodiments, R³ is chosen from phenyl and biphenyl, either of which may be optionally substituted with between 0 and 3 R⁶ groups.

In some embodiments,
m is an integer from 0 to 1;
Y is chosen from NR^{4a}, O, S, SO₂, and CH₂;
n is an integer from 1 to 3; and
R^{4a} is chosen from hydrogen and alkyl.

In some embodiments,
m is 0;
Y is CH₂; and
n is an integer from 1 to 3.

In some embodiments, n is 1. In some embodiments, n is 2. In some embodiments, n is 3.

In some embodiments, R⁶ is chosen from lower alkyl, halogen, lower alkoxy, OCF₃ and CF₃.

In some embodiments, R⁴ is hydrogen.

In some embodiments, R⁴ is methyl.

In some embodiments, n is 2 or 3.

In some embodiments, the nitrogen-containing heterocycloalkyl or heteroaryl ring formed by R¹ and R² together with the nitrogen to which they are attached contains 3 to eight atoms.

In some embodiments, R¹ and R² are taken together to form a nitrogen-containing heterocycloalkyl, which may be optionally substituted with between 0 and 3 R⁶ groups.

In some embodiments, the nitrogen-containing heterocycloalkyl formed by R¹ and R² together with the nitrogen to which they are attached is chosen from:

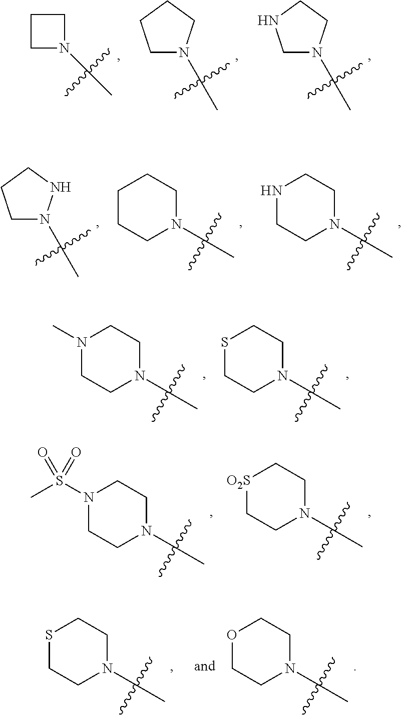

In some embodiments, the nitrogen-containing heterocycloalkyl formed by R¹ and R² together with the nitrogen to which they are attached is chosen from:

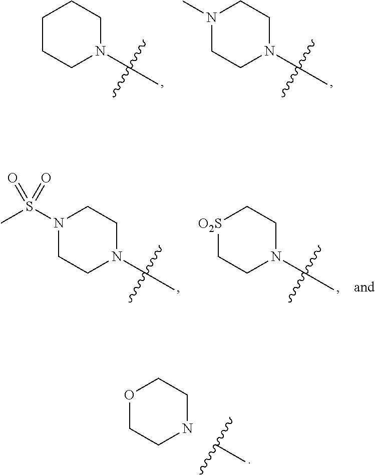

In some embodiments, the nitrogen-containing heterocycloalkyl formed by R¹ and

R² together with the nitrogen to which they are attached is

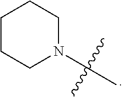 [SMILES: *N1CCCCC1]

In some embodiments, the nitrogen-containing heterocycloalkyl formed by R¹ and R² together with the nitrogen to
which they are attached is

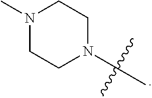 [SMILES: *N1CCN(C)CC1]

In some embodiments, the nitrogen-containing heterocycloalkyl formed by R¹ and R² together with the nitrogen to which they are attached is

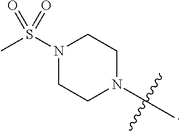 [SMILES: *N1CCN(S(C)(=O)=O)CC1]

In some embodiments, the nitrogen-containing heterocycloalkyl formed by R¹ and R² together with the nitrogen to which they are attached is

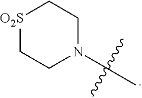 [SMILES: *N1CCS(=O)(=O)CC1]

In some embodiments, the nitrogen-containing heterocycloalkyl formed by R¹ and R² together with the nitrogen to which they are attached is

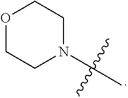 [SMILES: *N1CCOCC1]

In some embodiments, n is 2 or 3.

In some embodiments, R¹ and R² taken together form a nitrogen-containing heteroaryl, which may be optionally substituted with between 0 and 3 R⁶ groups.

In some embodiments, the nitrogen-containing heteroaryl is chosen from pyrrole, imidazole, and pyrazole.

In some embodiments, R⁵ is aryl, which may be optionally substituted with between 0 and 3 R⁶ groups, each of which is independently chosen from lower alkyl, halogen, lower alkoxy, OCF₃ and CF₃.

In some embodiments, R⁵ is phenyl, which may be optionally substituted with between 0 and 3 R⁶ groups, each of which is independently chosen from lower alkyl, halogen, lower alkoxy, OCF₃ and CF₃.

In some embodiments, n is 2 or 3.

In some embodiments, R⁵ is heteroaryl, which may be optionally substituted with between 0 and 3 R⁶ groups.

In some embodiments, R⁵ is a 5-6 membered monocyclic or 8-12 membered bicyclic heteroaryl, in which between one and five ring members may be heteroatoms chosen from N, O, and S, and which may be optionally substituted with between 0 and 3 R⁶ groups, each of which is independently chosen from lower alkyl, halogen, lower alkoxy, OCF₃ and CF₃.

In some embodiments, R⁵ is a 5-6 membered monocyclic heteroaryl, in which between one and five ring members may be heteroatoms chosen from N, O, and S, and which may be optionally substituted with 1 or 2 R⁶ groups, each of which is independently, if present, a lower alkyl groups.

In some embodiments, R⁵ is chosen from:

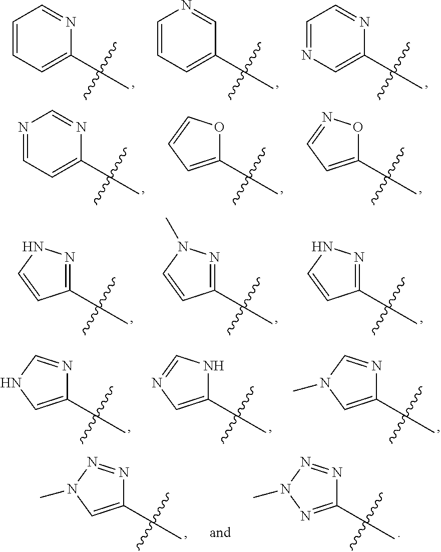

In some embodiments, wherein n is 2 or 3.

Some embodiments of the invention use compounds of the formula (IV):

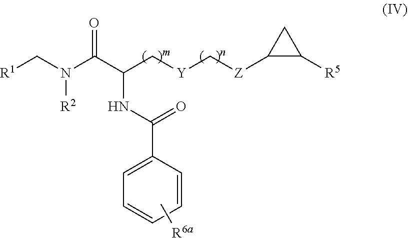

or a salt, polymorph, or solvate thereof, wherein:
Y is chosen from a bond, NR^{4a}, O, C(O)NH, NHC(O), S, SO₂, CHOH, and CH₂;
Z is chosen from a bond, NR^{4b}, O, C(O)NH, NHC(O), S, SO₂, and CH₂;
m is chosen from 0, 1, 2, 3, 4, and 5;
n is chosen from 0, 1, 2, and 3;
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4a} and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
R^{6a} is chosen from heteroaryl, cyano, and S(O)₂N(CH₃)₂;
each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;
with the proviso that when Y═CH₂ and Z═R^{4b}, then m+n≠3.

In some embodiments, Z in Formula IV is NR^{4b}.

In some embodiments of Formula IV, R^{4b} is chosen from methyl and hydrogen.

In some embodiments of Formula IV, R^{4b} is hydrogen.

In some embodiments of Formula IV, the alkyl, whether by itself or as a named part of another non-cyclic substituent, is C₁-C₈ alkyl.

In some embodiments of Formula IV, m is 0; Y is CH₂; and n is chosen from 0, 1, and 2. In some embodiments, n is 2.

In some embodiments of Formula IV, R¹ and R² are each independently chosen from alkyl, aminoalkyl, alkylsulfonylalkyl, alkoxyalkyl, and heteroaryl, and R¹ and R², together with the nitrogen to which they attach, form a nitrogen-containing heterocycloalkyl or heteroaryl ring, which may be optionally substituted with between 0 and 3 R⁶ groups.

In some embodiments of Formula IV, the nitrogen-containing heterocycloalkyl or heteroaryl ring formed by R¹ and R² together with the nitrogen to which they are attached contains 3 to eight atoms.

In some embodiments of Formula IV, R¹ and R² are taken together to form a nitrogen-containing heterocycloalkyl, which may be optionally substituted with between 0 and 3 R⁶ groups.

In some embodiments of Formula IV, the nitrogen-containing heterocycloalkyl is optionally substituted with between 0 and 3 R⁶ groups chosen from alkyl, halogen, CONH₂, SO₂CH₃, cyano, spiro-heterocycloalkyl, and oxo.

In some embodiments of Formula IV, the nitrogen-containing heterocycloalkyl is chosen from:

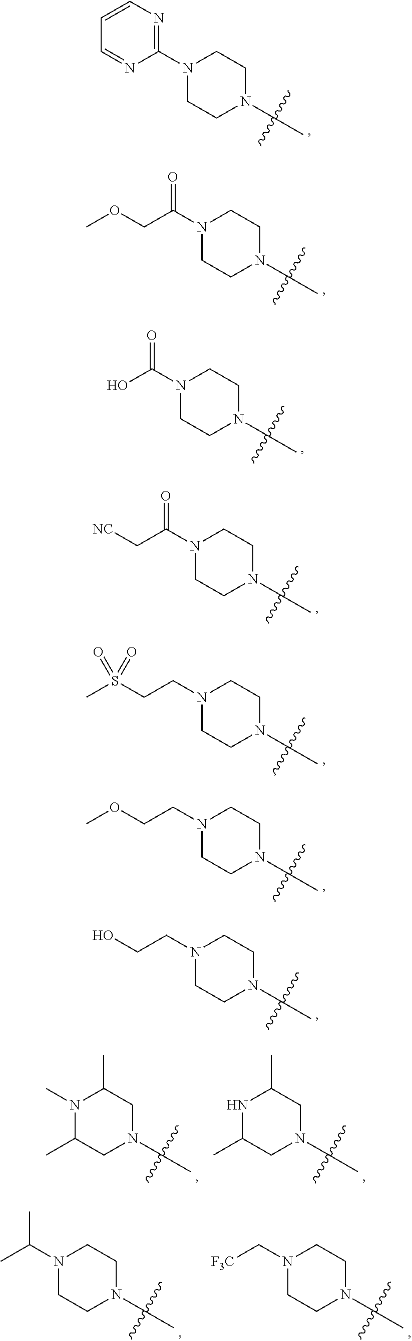

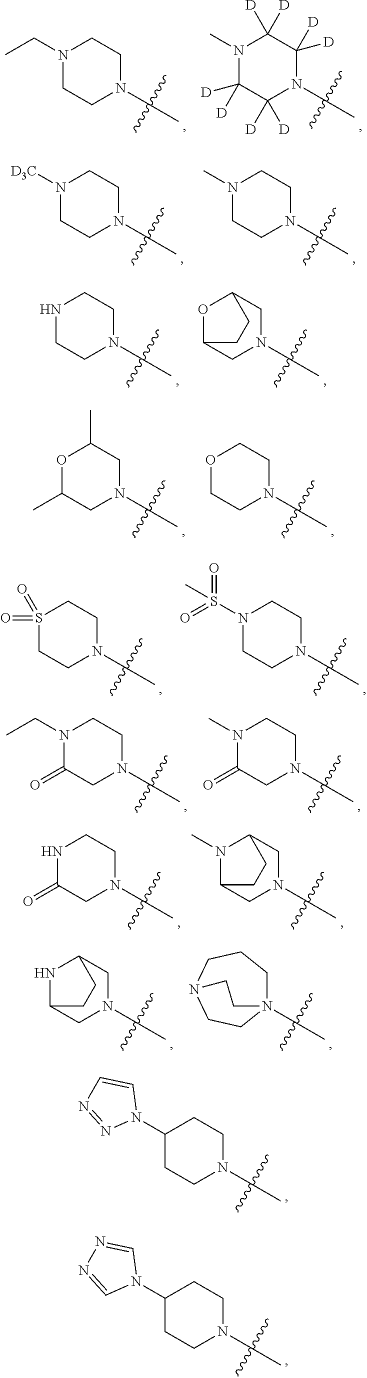

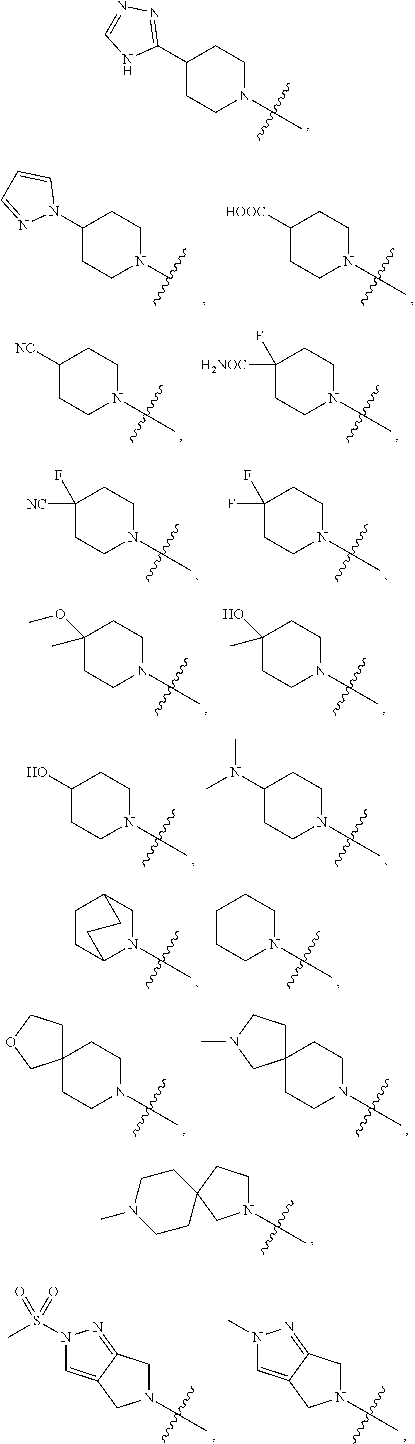

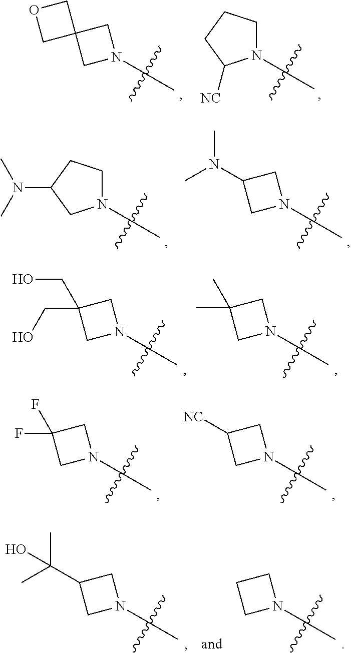

In some embodiments of Formula IV, the nitrogen-containing heterocycloalkyl is chosen from:

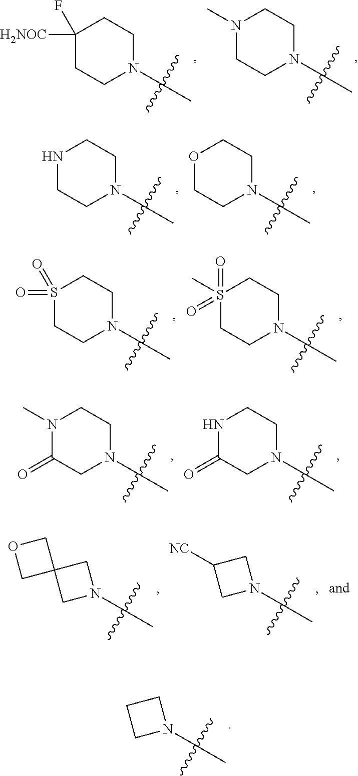

In some embodiments of Formula IV, the nitrogen-containing heterocycloalkyl is:

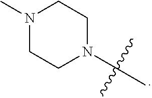 [SMILES: *N1CCN(C)CC1]

In some embodiments of Formula IV, each R^{6a} is chosen from cyano, S(O)₂N(CH₃)₂,

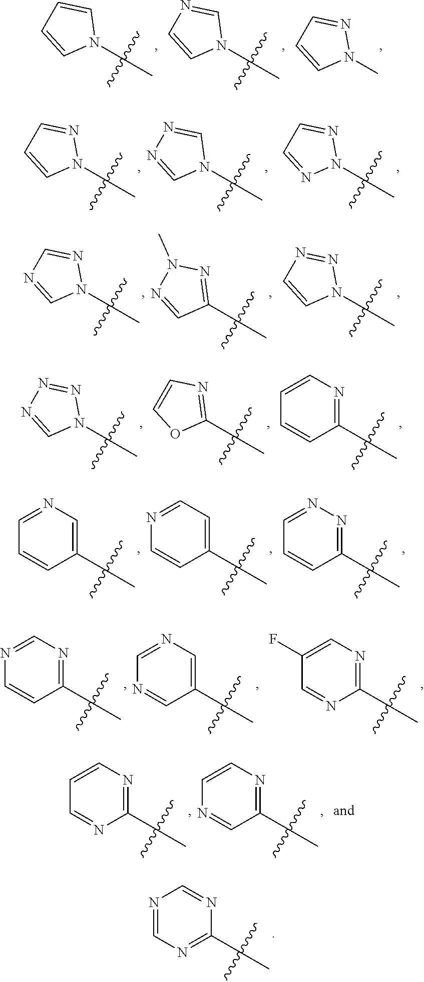

In some embodiments of Formula IV, R⁵ is phenyl, which may be optionally substituted with between 0 and 3 R⁶ groups.

In some embodiments of Formula IV, wherein R⁵ is:

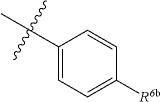

wherein R^{6b} is chosen from halogen, hydroxy, and methoxy.

In some embodiments of Formula IV, R^{6b} is chosen from fluoro, methoxy, and hydroxy.

In some embodiments of Formula IV, R^{6b} is fluoro.

In some embodiments of Formula IV, the LSD1 inhibitor of Formula IV is a compound of Formula V:

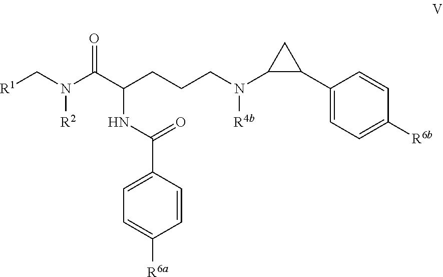

or a salt, polymorph, or solvate thereof, wherein:
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4a} is chosen from hydrogen, alkyl, alkenyl, alkynyl, and cycloalkyl;
R^{6a} is chosen from heteroaryl, cyano, and S(O)₂N(CH₃)₂;
each R⁶ and R^{6b} is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl.

In some embodiments of Formula V, R^{4b} is chosen from methyl and hydrogen.

In some embodiments of Formula V, R^{4b} is hydrogen.

In some embodiments of Formula V, each R^{6a} is chosen from cyano,

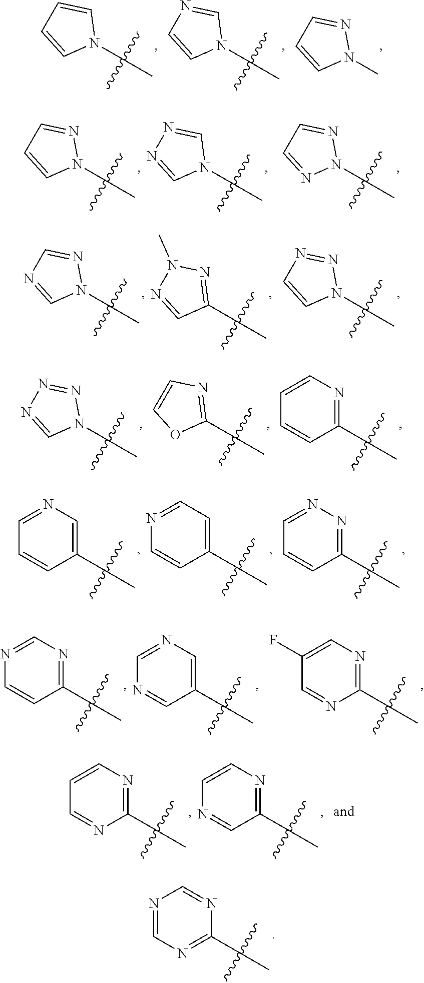

In some embodiments of Formula V, R¹ and R² are each independently chosen from alkyl, aminoalkyl, alkylsulfonylalkyl, alkoxyalkyl, and heteroaryl, and R¹ and R², together with the nitrogen to which they attach, form a nitrogen-containing heterocycloalkyl or heteroaryl ring, which may be optionally substituted with between 0 and 3 R⁶ groups.

In some embodiments of Formula V, the nitrogen-containing heterocycloalkyl or heteroaryl ring formed by R¹ and R² together with the nitrogen to which they are attached contains 3 to eight atoms.

In some embodiments of Formula V, R¹ and R² are taken together to form a nitrogen-containing heterocycloalkyl, which may be optionally substituted with between 0 and 3 R⁶ groups.

In some embodiments of Formula V, the nitrogen-containing heterocycloalkyl is chosen from:

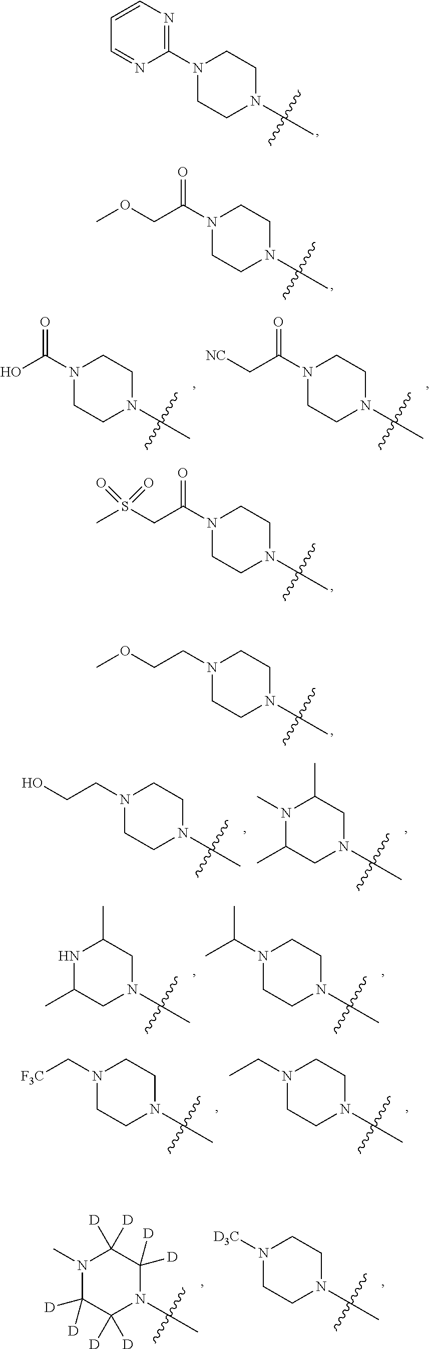

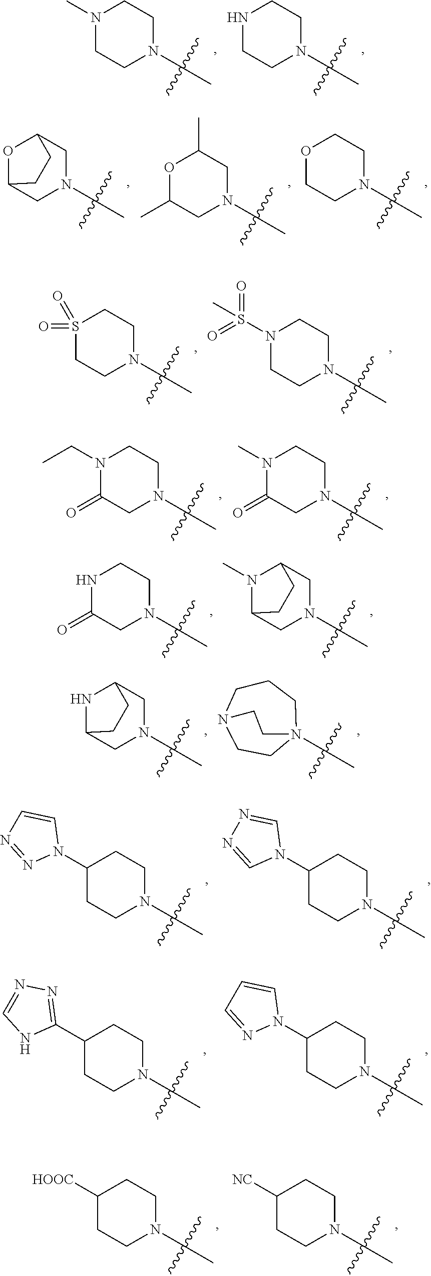

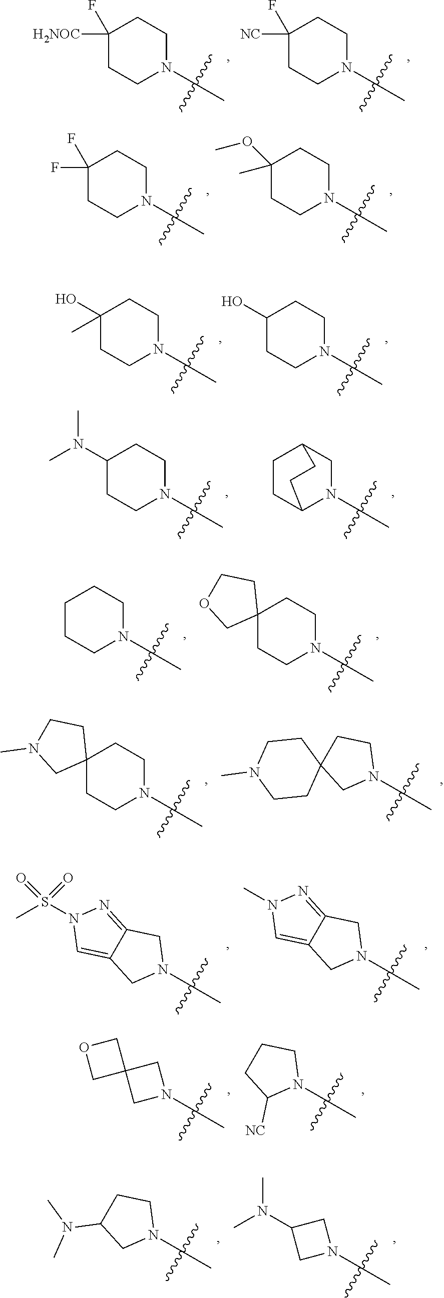

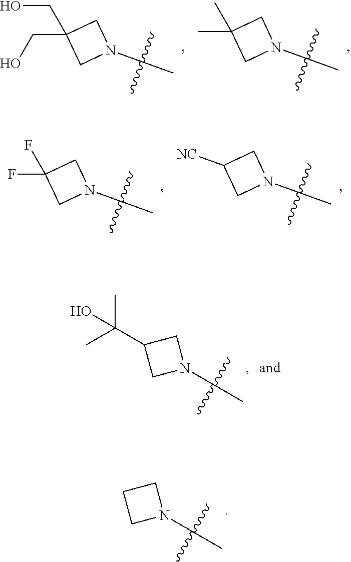

In some embodiments of Formula V, the nitrogen-containing heterocycloalkyl is chosen from:

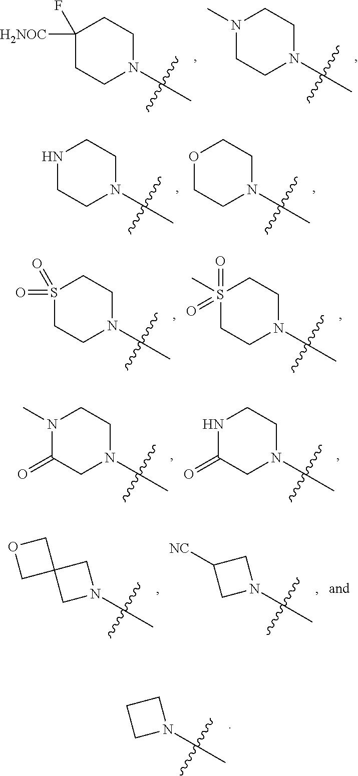

In some embodiments of Formula V, the nitrogen-containing heterocycloalkyl is:

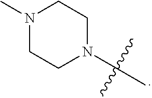 [SMILES: *N1CCN(C)CC1]

In some embodiments of Formula V, R^{6b} is chosen from fluoro, methoxy, and hydroxy.

In some embodiments of Formula V, R^{6b} is fluoro.

In some embodiments, the LSD1 inhibitor of Formula V is:

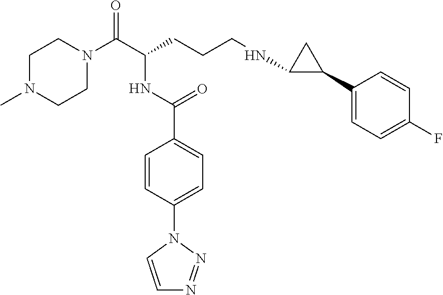 [SMILES: CN1CCN(C(=O)[C@H](CCCN[C@@H]2C[C@H]2c2ccc(F)cc2)NC(=O)c2ccc(-n3ccnn3)cc2)CC1]

or a salt, polymorph, or solvate thereof.

In some embodiments, the LSD1 inhibitor of Formula V is a salt of Formula VI:

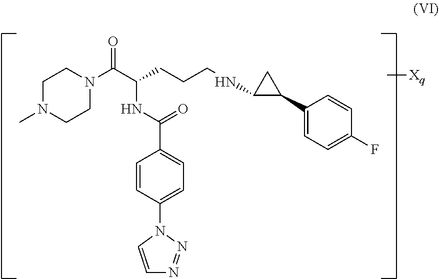

or a polymorph or solvate thereof, wherein:
X is chosen from tosylate, sulfate, tartrate, oxalate, besylate, fumarate, citric, esylate, and malate; and
q is an integer chosen from 1 and 2.

In some embodiments, X is tosylate.

In some embodiments, q is 2.

In some embodiments, the LSD1 inhibitor is a compound recited in WO2016130952 or WO2017079753.

In some embodiments, the LSD1 inhibitor is bomedemstat (IMG-7289), N-[(2S)-5-{[(1R,2S)-2-(4-fluorophenyl)cyclopropyl]amino}-1-(4-methylpiperazin-1-yl)-1-oxopentan-2-yl]-4-(1H-1,2,3-triazol-1-yl)benzamide, bis-tosylate salt,

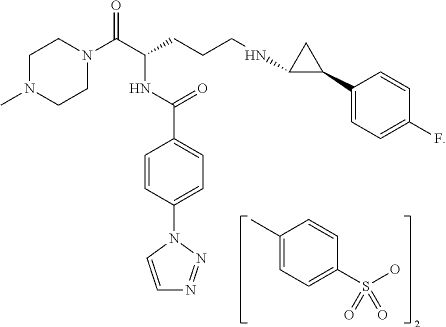

In some embodiments, the LSD1 inhibitor is chosen from bomedemstat (IMG-7289), iadademstat (ORY-1001), vafidemstat (ORY-2001), pulrodemstat (CC-90011), seclidemstat (SP-2577), INCB059872, TAS1440, SYHA1807, RO7051790, GSK2879552, MC2580, and DDP-38003.

The compounds above may be synthesized as disclosed in US20160237043, WO2016130952, WO2017079753, WO2018035259, and WO2018035249.

Additional LSD1 inhibitors useful in the methods disclosed herein include those disclosed in the following references:

| | | |
|---|---|---|
| WO2014/164867A1 | WO2012/042042A1 | US2015/0225375A1 |
| WO2015/021128A1 | WO2012/045883A1 | US2015/0225379A1 |
| WO2016/130952A1 | WO2012/072713A2 | US2016/0009711A1 |
| U.S. Pat. No. 9,388,123 | WO2012/107499A1 | US2016/0009712A1 |
| WO2010/143582A1 | WO2012/107498A1 | US2016/0009720A1 |
| WO2012/135113A2 | WO2012/156537A2 | US2016/0009721A1 |
| WO2009/027349A2 | WO2012/156531A2 | WO2016/161282A1 |
| WO2010/043721A1 | WO2013/057320A1 | US2015/0315126A1 |
| WO2010/084160A1 | WO2013/057322A1 | US2015/0065495A1 |
| WO2011/035941A1 | U.S. Pat. No. 8,765,820 | WO2015/120281A1 |
| WO2011/042217A1 | U.S. Pat. No. 8,389,580 | WO2016/123387A1 |
| WO2011/106105A2 | US2016/0120862A1 | WO2016/130952 |
| WO2011/106106A2 | US2015/0025054A1 | WO2016172496A1 |
| WO2011/106573A2 | WO2015/134973A1 | US2015/0065495A1 |
| WO2011/106574A2 | US2014/0011857A1 | WO2017/079753 |
| WO2011/131697A1 | US2012/0322877A1 | |
| WO2012/013728A1 | US2015/0225394A1 | |
| WO2012/013727A1 | US2015/0225401A1 | |

the contents of which are hereby incorporated by reference as if written herein in their entireties.

### Compositions and Methods of Treatment

Also provided herein is a therapeutic composition comprising a population of CAR-bearing immune effector cells according to the fourth aspect of the invention and at least one therapeutically acceptable diluent, carrier and/or adjuvant.

Also provided herein is a method for treatment of cancer in a patient comprising administering a population of CAR-bearing immune effector cells according to the third aspect of the invention or a therapeutic composition according to the fourth aspect of the invention to a cancer patient. In some embodiments, the immune effector cell or a population of CAR-bearing immune effector cells is a CAR-T cell or a population of CAR-T cells, according to the third aspect of the invention.

In some embodiments, the cancer is a hematologic malignancy. In some embodiments, the hematologic malignancy is a T-cell malignancy. In some embodiments, the T cell malignancy is T-cell acute lymphoblastic leukemia (T-ALL). In some embodiments, the T cell malignancy is non-Hodgkin's lymphoma. In some embodiments, the T cell malignancy is T-cell chronic lymphocytic leukemia (T-CLL). In some embodiments, the T cell malignancy is selected from T-cell acute lymphoblastic leukemia/lymphoma (T-ALL), human T-cell leukemia virus type 1-positive (HTLV-1+) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotropic intestinal T-cell lymphoma, Mycosis fungoides, Nodal peripheral T-cell lymphoma with TFH phenotype, Peripheral T-cell lymphoma (PTCL), NOS, Primary cutaneous γδ T-cell lymphoma, Primary cutaneous CD8+ aggressive epidermotropic cytotoxic T-cell lymphoma, Primary cutaneous acral CD8+ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

In some embodiments, the hematologic malignancy is a plasma cell malignancy. In some embodiments, the plasma cell malignancy is selected from lymphoplasmacytic lymphoma, plasmacytoma and multiple myeloma.

In some embodiments, the hematologic malignancy is a B-cell cell malignancy. In some embodiments, the B-cell malignancy is selected from wherein the B-cell malignancy is selected from diffuse large B cell lymphoma (DLBCL), chronic lymphocytic leukemia (CLL)/small lymphocytic lymphoma (SLL), and B cell-precursor acute lymphoblastic leukemia (ALL).

### Definitions

Unless specifically defined herein, all technical and scientific terms used have the same meaning as commonly understood by a skilled artisan in the fields of gene therapy, biochemistry, genetics, immunology, and molecular biology. All disclosed compositions and methods similar or equivalent to those described herein can be used in the practice or testing of the present disclosure.

The term “antigen” refers to a molecular entity that may be soluble or cell membrane bound in particular but not restricted to molecular entities that can be recognized by means of the adaptive immune system including but not restricted to antibodies or TCRs, or engineered molecules including but not restricted to transgenic TCRs, chimeric antigen receptors (CARs), scFvs or multimers thereof, Fab-fragments or multimers thereof, antibodies or multimers thereof, single chain antibodies or multimers thereof, or any other molecule that can execute binding to a structure with high affinity.

“Antigen binding domain” means an oligo- or polypeptide that is capable of binding a ligand. Generally, the antigen binding domain will be capable of interacting with a cell surface molecule which may be an antigen, a receptor, a peptide ligand, a protein ligand of the target, or a polypeptide of the target. The extracellular ligand-binding domain can specifically bind to an antigen with an affinity constant or affinity of interaction (K_{D}) from about 0.1 μM to about 100 nM, for example from about 0.1 μM to about 10 μM, or from about 0.1 μM to about 1 μM. Methods for determining the affinity constant or affinity of interaction (K_{D}) are well-known in the art. In some instances, the extracellular ligand-binding domain is chosen to recognize a ligand that acts as a cell surface marker on target cells associated with particular disease states. In some embodiments, the antigen binding domain comprises a single chain antibody fragment (scFv) comprising a V_{H} chain, a peptide linker and a V_{L} chain. In some embodiments, the peptide linker between the V_{H} and V_{L} chains has the amino acid sequence GGGGS₍₁₋₆₎, for example GGGGS₍₁₋₄₎.

“Biologically active fragment” generally means a fragment of a polypeptide that substantially retains the biological activity of the intact polypeptide in the context of CAR function.

The term “cancer” is known medically as a malignant neoplasm. Cancer is a broad group of diseases involving upregulated cell growth. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

A “CAR-bearing immune effector cell” is an immune effector cell which has been transduced with at least one CAR. A “CAR-T cell” is a T cell which has been transduced with at least one CAR; CAR-T cells can be mono, dual, or tandem CAR-T cells. CAR-T cells can be autologous, meaning that they are engineered from a subject's own cells, or allogeneic, meaning that the cells are sourced from a healthy donor, and in many cases, engineered so as not to provoke a host-vs-graft or graft-vs-host reaction. Donor cells may also be sourced from cord blood or generated from induced pluripotent stem cells.

“CAR-T cell” means a T cell which expresses a chimeric antigen receptor. The T cell expressing a CAR molecule may be a helper T cell, a cytotoxic T cell, a viral-specific cytotoxic T cell, a memory T cell, or a gamma delta (γδ) T cell.

“CD8a leader”, “CD8α leader”, “CD8a leader polypeptide” or “CD8α leader polypeptide” means the CD8a protein or a biologically fragment thereof that functions to initiate translocation of the extracellular domain of the CAR through the immune effector cell membrane to its membrane surface. A biologically active fragment in this case is defined as a fragment of at least 10 amino acids of the CD8α signal peptide that directs the appended polypeptide to the cell membrane and/or cell surface. Examples of functional fragments of the human CD80 signal peptide are given in Table 1.

“Chimeric antigen receptor” (“CAR”) means a recombinant fusion protein comprising: 1) an extracellular ligand-binding domain, also referred to herein as an antigen binding domain an antigen-domain, 2) a transmembrane domain, and 3) a signaling transducing domain.

“Co-stimulatory domain” means an oligopeptide or a polypeptide that enhances the signaling capability of a signaling domain.

“Deficient in at least one or more antigens to which the one or more CARs specifically binds” means the corollary antigen of the immune effector cell to which the antigen binding domain binds is modified such that the chimeric antigen receptor no longer specifically binds the modified antigen. Stated differently, a cell that “is deficient in” an antigen is one that does not express, expresses a reduced amount of, or expresses an altered type of, the antigen, such that chimeric antigen receptors or other proteins which would typically bind the antigen have a reduced or eliminated ability to do so.

A “dual” CAR-bearing immune effector cell, for example a “dual CAR-T cell” or a “dual CAR-NK cell” means an engineered immune effector cell (e.g., T or NK cell) with two distinct chimeric antigen receptor polypeptides with affinity to different target antigen expressed within the same effector cell, wherein each CAR functions independently. The CAR may be expressed from single or multiple polynucleotide sequences.

“Fratricide” means a process which occurs when a CAR-bearing immune effector cell becomes the target of, and is killed by, another CAR-bearing immune effector cell comprising the same chimeric antigen receptor as the target of the CAR-bearing immune effector cell, because the targeted cell expresses the antigen specifically recognized by the chimeric antigen receptor on both cells.

As used herein, the phrase “from a proximal N-terminus to a distal C-terminus,” when used to describe elements comprised in a chimeric antigen receptor, does not exclude variations known to those of skill in the art and linking sequences between the elements.

“Hinge” means any oligo- or polypeptide that functions to link the transmembrane domain to the antigen binding domain. In particular, the hinge is used to provide more flexibility and accessibility for the antigen binding domain.

“Immediately distal to” means in proximity on the C-terminal side of an element, such as a linker or biologically functional domain. It can mean contiguous with, or in such close proximity as to not interfere with its own function, of the function of the element, or their combined function.

“Immediately proximal to” means in proximity on the N-terminal side of an element, such as a linker or biologically functional domain. It can mean contiguous with, or in such close proximity as to not interfere with its own function, of the function of the element, or their combined function.

“The term “immune cell” or “immune effector cell” refers to a cell that may be part of the immune system and executes a particular effector function such as alpha-beta T cells, NK cells, NKT cells (including iNKT cells), LAK gamma-delta T cells. cells, B cells, innate lymphoid cells (ILC), mesenchymal stem cells or mesenchymal stromal cells (MSC), monocytes and macrophages. Preferred immune cells are cells with cytotoxic effector function such as alpha-beta T cells, NK cells, NKT cells (including iNKT cells), ILC, CIK cells, LAK cells, and gamma-delta T cells. “Effector function” means a specialized function of a cell, e.g., in a T cell an effector function may be cytolytic activity or helper activity including the secretion of cytokines.

The term “malignant” or “malignancy” describes cells, groups of cells or tissues that constitute a neoplasm, are derived from a neoplasm or can be the origin of new neoplastic cells. The term is used to describe neoplastic cells in contrast to normal or healthy cells of a tissue. A malignant tumor contrasts with a non-cancerous benign tumor in that a malignancy is not self-limited in its growth, is capable of invading into adjacent tissues, and may be capable of spreading to distant tissues. A benign tumor has none of those properties. Malignancy is characterized by anaplasia, invasiveness, and metastasis as well as genome instability. The term “premalignant cells” refer to cells or tissue that is not yet malignant but is poised to become malignant.

As used herein, the term “sequence identity” means the percentage of identical nucleotide or amino acid residues at corresponding positions in two or more sequences when the sequences are aligned to maximize sequence matching, i.e., taking into account gaps and insertions. Identity can be readily calculated by known methods. Methods to determine identity are designed to give the largest match between the sequences tested. Moreover, methods to determine identity are codified in publicly available computer programs. Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc. See generally, Altschul, S. F. et al., J. Mol. Biol. 215:403-410 (1990) and Altschul et al. Nucl. Acids Res. 25:3389-3402 (1997). One example of an algorithm that is suitable for determining percent sequence identity and sequence similarity is the BLAST algorithm.

“Signaling domain” or “signal transducing domain” means a signal transducing domain or intracellular signaling domain of a CAR which is responsible for intracellular signaling following the binding of the antigen binding domain to the target resulting in the activation of the immune cell and immune response. In other words, the signal transducing domain is responsible for the activation of at least one of the normal effector functions of the immune cell in which the CAR is expressed.

The terms “specifically binds” or “specific for” or “specifically recognize” with respect to an antigen-recognizing receptor refer to an antigen-binding domain of said antigen-recognizing receptor which recognizes and binds to a specific polymorphic variant of an antigen, but does not substantially recognize or bind other variants.

“Suicide gene” is a gene that causes an immune effector cell to undergo cell death when the cell exceeds a certain level of immune effector activity. Suitable suicide gene systems known in the art include, but are not limited to, several herpes simplex virus thymidine kinase (HSVtk)/ganciclovir (GCV) or inducible caspase 9 proteins. In one embodiment, the suicide gene is a chimeric CD34/thymidine kinase.

A “tandem” CAR-bearing immune effector cell, for example a “tandem CAR-T cell” or a “tandem CAR-NK cell” means an engineered immune effector cell (e.g., T or NK cell) with a single chimeric antigen polypeptide comprising two or more distinct extracellular ligand-binding domains capable of interacting with two or more different cell surface molecules, wherein extracellular ligand-binding domains are linked together by a flexible linker and share one or more effector/costimulatory domains, wherein the binding of the first or the second extracellular ligand-binding domain will signal through one or more the costimulatory domains and a signaling transducing domain. A tandem CAR may be bispecific or trispecific.

“Therapeutic composition” refers to substances which are suitable for use in contact with the tissues of patients without undue toxicity, irritation, and allergic response, are commensurate with a reasonable benefit/risk ratio, and/or are effective for their intended use.

“Transmembrane domain” means a generally hydrophobic peptide or polypeptide that traverses the cell membrane, anchors the CAR to the T cell surface, and connects the antigen-binding domain to the signaling transducing domain, impacting the expression of the CAR on the T cell surface. The distinguishing feature of the transmembrane domain in the present disclosure is the ability to be expressed at the surface of an immune cell to direct an immune cell response against a pre-defined target cell.

“Truncated CD34 domain” or “TrCD34 domain” means a fragment of CD34 polypeptide.

### EXAMPLES

The following examples are intended to further illustrate certain non-limiting embodiments of the invention and are not intended to limit in any way the scope of the invention.

### Example 1—Method of Making CAR-NK Cells

In brief, NK cells are purified from normal donor PBMC and incubated in IL-Dec. 15, 2018. The cytokines are washed away and the cells are then incubated in high dose IL-15 and transduced with CAR lentivirus, 2×. Cells were allowed to rest (in vivo or in vitro) and assessed for enhanced effector functions. A more detailed description of an exemplary procedure is given below.

Primary NK cells may be purified from peripheral blood mononuclear cells (PBMCs). A PBMC is any peripheral blood cell having a round nucleus. These cells consist of lymphocytes (T cells, B cells, NK cells) and monocytes, whereas erythrocytes and platelets have no nuclei, and granulocytes (neutrophils, basophils, and eosinophils) have multi-lobed nuclei. Other products were derived from stem cells or cell lines.

Cytokine addition may be important for NK cell production. IL-Dec. 15, 2018, or fusion proteins comprising functional fragments of such cytokines, may be added. Cytokines may then be washed away and cells cultured in IL-15 for the remainder of the assay.

Additionally, NK cells are sensitive to polybrene; as such, polybrene is not used during the transduction, which is conventionally used.

### Construction of Lentiviral Vector and Transduction of NK Cells

CAR may be introduced using lentivirus. The cassette encoding the chimeric antigen receptor (CAR) may be incorporated into, e.g., a MND lentiviral backbone to generate the lentiviral vector. To produce the lentiviral supernatant, 293T cells may be cotransfected with lentiviral vectors, pMND-G, pMND-Lg, and pMDN-REV, using the Calcium Chloride transfection reagent, and supernatant containing the lentivirus collected 24-48 and hours later and concentrated using ultracentrifugation. For transduction, purified cytokine-activated (IL-Dec. 15, 2018) NK cells may be plated in complete culture media supplemented with 50 ng/ml IL-15. Viral supernatant may be added to the cells, which may be spinfected at 2000 rpm for 90 m at room temperature. The cells can be incubated at 37° C. in 5% CO2. To maximize viral transduction efficacy, cells may be spinfected on days 1 and 2. The cells are then washed and used immediately or cultured in complete media supplemented with 1 ng/mL IL-15. Maximum vector expression is expected by day 7.

### Example 2—Method of Making CAR-T Cells

CAR-bearing immune effector cells may be constructed, optionally with a genome editing step to effect deletion or suppression of one or more surface proteins. Such surface proteins many include, for example, those that form part of the TCR complex, which may induce GvHD if the cells are administered to patients in the allogeneic setting, or those that are the target antigen of the CAR, which may induce fratricide if expression of the antigen on CAR-T is not suppressed.

For example, in one protocol, on Day 0, CD4+CD8+ T cells are thawed in a cell culture media. The required number of cells are centrifuged at 200×g for 10 minutes at room temperature. Supernatant is removed completely, cells resuspended cell culture media (TexMacs) supplemented with IL-7 (10 ng/ml) and IL-15 (10 ng/ml) at concentration of 1×10⁶/ml. T cells are stimulated with Miltenyi research grade TransAct™ (10 μl/ml).

On day 1, the required amount of viral vector comprising CAR is added to the activated cells at the required M.O.I (Multiplicity of Infection). Cells and virus are mixed and placed back in incubator at 37° C.

On day 3, activated cells are washed to remove stimulation.

If genome editing is desired, cells are harvested and counted. The required number of cells are centrifuged at 100×g for 10 minutes at room temperature. Supernatant is removed completely, cells resuspended in Electroporation buffer (1 ml) (e.g., Maxcyte EP buffer) and transferred to a microcentrifuge tube, and centrifuged at 100×g for 10 minutes at room temperature. Supernatant is removed completely, and cells then resuspended in electroporation buffer (e.g., MaxCyte EP buffer), at the desired concentration (e.g., 5×10⁷/ml).

Commercially available Cas9 Protein (10 μg) and commercially synthesized gRNA (20 μg) are complexed at room temperature for 10 minutes.

Cells (100 μl) are transferred to the tube containing complexed Cas9/gRNA, gently mixed, and everything transferred into a MaxCyte OC100 cuvette. Electroporation is thereafter commenced using Maxcyte program Expanded T cell 2. After this procedure, the activated cells may be transferred to 10 ml of pre-warmed media and returned to the incubator to expand for an additional 7-12 days.

FACS analysis may be used to show the purity of CAR-transduced cells (CAR expression and target gene deletion).

### Example 3—Viral Vector Production

Lentivirus (e.g., comprising expression cassette for CAR and any tags) may be produced using the Lenti-X 293T cell line (Takara Bio, Mountain View, CA). The Lenti-X 293T cells may be transfected with the CAR lentiviral vector and packaging plasmids, pMD.Lg/rRRE, pMD.G, and pRSV.REV14, using Lipofectamine 2000 Transfection Reagent from Invitrogen according to the manufacturer's instructions. 36 hours following transfection the viral supernatant may be harvested, filtered to remove cell debris, and concentrated via ultracentrifugation at 4° C. for 90 minutes at 25,000 rpm (Optima LE-80K Ultracentrifuge, Beckman Coulter, Indianapolis IN). Once concentrated, the virus is re-suspended in phosphate buffered saline.

### Example 4—CRISPR/Cas9 Editing to Remove Genes

Optionally, T cells may be cultured in Xcyte media supplemented with 50 U/mL IL-2, 10 ng/ml IL-2, 10 ng/ml IL-15, and 10 ng/ml IL7 in the presence of anti-CD3/CD28 beads (bead to cell ratio 3:1). On day +2 post activation, stimulation is removed and 1×10⁷ T cells electroporated in 100 μl MaxCyte buffer containing 15 μg spCas9 (Trilink CA.) and 20 μg of each gRNA (one or multiple, for multiplex editing; appropriate gRNA sequences for target genes are available in the art) using, e.g., a MaxCyte GT. The cells may then be transduced with lentiviral particles in the presence of polybrene (Sigma Aldrich) (final concentration 6 μg/ml) on day +3. The cells are then expanded for, e.g., 6 days following transduction before being used in later experiments.

Targeted deep sequencing may be used to confirm editing. The locus of the gene targeted by CRISPR editing may be amplified with primers. PCR products are sequenced using, e.g., the Illumina MiSeq platform (San Diego CA). Editing efficiencies may be determined as a percentage of sequencing reads with indels aligned to reads obtained from WT cells.

### Example 5—Suppression/Reversal of Terminal Differentiation in CAR-T Cells

To mimic the tumor microenvironment (TMA) that continuously exposes antitumor T cells to the tumor antigen, CAR-T cells may be repeatedly stimulated with target cells (e.g., CD19 CAR-T cells may be stimulated with CD19⁺ K562-CD19 or NALM6 cells, which express CD19) at different intervals. These expanded CAR-T cells typically nearly lose a CD45RA⁺ CD62L⁺ CCR7⁺ CD27⁺ CD28⁺ T-cell population (stem cell-like memory T cells: T_{SCM}) and include a small population of CD45RA-CD62L⁺ CCR7⁺ CD27⁺ CD28⁺ cells, representing a central memory phenotype (T_{CM}). CAR-T cells stimulated by cells expressing target antigen (e.g., K562-CD19) express higher levels of T-cell exhaustion markers such as PD1, LAG3, and TIM3 than those stimulated by NALM6, perhaps due to high antigen load, and lose polyfunctional cytokine production capacity.

CD19 CAR-T cells that are genetically modified to delete PRDM1 using CRISPR/Cas9 and subjected to this protocol lose Blimp1 expression and exhibit improved maintenance of T_{SCM} and T_{CM} phenotypes, including a higher frequency of CD45RA⁺/2CD62L⁺ CCR7⁺ CD27⁺ CD28⁺ IL7R⁺ cells, as well as elevated TOX expression and cytokine production (e.g., TNFα and IL-2). See, Yoshikawa T et al., “Genetic ablation of PRDM1 in antitumor T cells enhances therapeutic efficacy of adoptive immunotherapy,” Blood 139 (14): 2156-72 (2022).

This protocol may be modified to include treatment with LSD1 inhibitor, in place of PRDM1 deletion. The inhibitor may be administered on various schedules and over a range of concentrations depending on the inhibitor; for example bomedemstat may be used. It is expected that treatment with LSD1 inhibitor will similarly preserve T_{SCM} and T_{CM} phenotypes, and that treatment with bomedemstat or another LDS1 inhibitor which inhibits both demethylase activity and the ability of Growth Factor Independent 1 and/or Growth Factor Independent 1b (GFI1 and/or GFI1b and Blimp1) to bind to LSD1 will outperform LSD1 inhibitors which do not have this dual activity.

### Example 6—Cell Lines for Cancer Cell Killing Assays, and Cell Line Preparation

Appropriate cell lines, e.g., K562-CD19 or NALM6 cells (for assaying CD19-targeting CAR comprising, e.g., the clone FMC63-derived scFv linked with CD28 costimulatory and CD3z signaling domains) may be obtained from commercial or public sources, e.g., DSMZ-German collection of Microorganisms and Cell Cultures (Leibniz, Germany) and/or ATCC Global Bioresource Center (Manassas VA). The cell lines are mycoplasma tested upon receipt. Cell lines may be transduced with GFP lentivirus, e.g., at an MOI of 5:1. Following transduction, the GFP positive cells are sorted and expanded to establish pure GFP-expressing cell lines.

### Example 7—Killing of Cells by CAR-T Cells: Chromium Release

CAR-T cells may be incubated with [⁵¹Cr]-labeled cell lines (e.g., 1×10⁴ total cells/well) at an effector:target [E:T] ratio ranging from, e.g., 25:1 to 0.25:1 in appropriate media. For example, K562-CD19 or NALM6 cells may be used for assaying CAR-T cells comprising CD19-targeting CAR comprising, e.g., the clone FMC63-derived scFv linked with CD28 costimulatory and CD3z signaling domains). The chromium release assay is performed according to a standard protocol. It is expected that treatment of tumor cells with a combination of CAR-T and LDS1i will result in improved, if somewhat delayed, cell killing compared to CAR-T alone, and that treatment with bomedemstat or another LDS1 inhibitor which inhibits both demethylase activity and the ability of Growth Factor Independent 1 and/or Growth Factor Independent 1b (GFI1 and/or GFI1b and Blimp1) to bind to LSD1 will outperform LSD1 inhibitors which do not have this dual activity.

### Example 8—Killing of Cells by CAR-T Cells: FACS

To evaluate the cytotoxic activity of CAR-T cells, 1×10⁵ CAR-T cells may be cocultured with the indicated ratio of EGFP1 target cells. The absolute counts of viable target cells are determined by flow cytometry. The frequency of surviving tumor cells is calculated as the ratio of the cell counts incubated without CAR-T cells. Moreover, dead cells are discriminated using the LIVE/DEAD Fixable Near-IR Dead Cell Stain Kit (Thermo Fisher Scientific). It is expected that treatment of tumor cells with a combination of CAR-T and LDS1i will result in improved, if somewhat delayed, cell killing compared to CAT-T alone, and that treatment with bomedemstat or another LDS1 inhibitor which inhibits both demethylase activity and the ability of Growth Factor Independent 1 and/or Growth Factor Independent 1b (GFI1 and/or GFI1b and Blimp1) to bind to LSD1 will outperform LSD1 inhibitors which do not have this dual activity.

### Example 9—T Cell Phenotype Analysis

Cultured T cells may be washed in PBS/0.1% BSA and re-suspended at 1×10⁶ cells in 50 uL Brilliant Buffer (BD Biosciences) supplemented with 4% rat serum for 15 minutes at 4 C. Cells were then incubated for 30 minutes at 4° C. in 100 μL of Brilliant Buffer using the following antibody fluorophore conjugates (all from BD Biosciences unless otherwise noted): CD2 BUV395, CD8 BUV496, CD3 BUV661, CD38 BV421, Tim-3 BV450 (BioLegend), PDL1 BV510 (BioLegend), PD1 BV605 (BioLegend), CD4 BV650, CD45 BV711, CD196/CCR6 (BioLegend), CD45RA, Cy5.5, CD183/CXCR3 PE, CD34 PE-Cy7 (BioLegend), Tigit APC (BioLegend), CD127 APC-eF780 (eBioscience), CD62L AF700 (BioLegend). Cells are then washed twice in PBS/0.1% BSA and data acquired on a ZE5 (Yeti) cytometer (BioRad/Propel Labs). Compensation and analyses are performed on FlowJo V10 (TreeStar) using fluorescence minus one (FMO) controls. Statistical analyses may be performed on GraphPad Prism 7.

### Example 10—Xenogeneic Mouse Model

The anti-leukemic effect of the combination of CAR-T cells and LSD1 inhibitors may be tested in vivo using NSG mice inoculated with cancer cells. Male mice approximately 4 to 10 weeks old are injected into the tail vein with, e.g., 5×10⁵ cells (e.g., K562-CD19 or NALM6 cells for CD19-CAR-Tcells) on day-5. CAR-T (2×10⁶)+LSD1 inhibitor, CAR-T (2×10⁶) alone, and controls are injected via the lateral tail vein on Day 0.

To track the tumor growth in vivo, mice are injected intraperitoneally with 50 μg/g D-luciferin (Biosynth, Itasca, IL, USA) and imaged by methods known in the art, at various points through, e.g., 80 days—for example, weekly, on days 7, 14, 21, 28, 35, etc. Persistence of CAR-T cells in vivo, and the fraction of the sampled CAR-T cells at each time point expressing TSCM and TCM phenotypes compared to the infused pool, may also be tracked.

Log-rank Mantel-Cox test may be used to determine significant difference in survival. Statistical analysis of tumor burden, determined by BLI imaging, may be determined using two-way ANOVA for repeated measurement data, followed by a step-down Bonferroni adjustment for multiple comparisons. BLI may be performed in a blinded fashion.

The combination of CAR-T cells and LSD1 inhibitors is expected to yield better control of tumor growth, better CAR-T cell persistence, and maintenance of T_{SCM} and T_{CM} phenotypes than administration of CAR-T cells alone, and that treatment with bomedemstat or another LDS1 inhibitor which inhibits both demethylase activity and the ability of Growth Factor Independent 1 and/or Growth Factor Independent 1b (GFI1 and/or GFI1b and Blimp1) to bind to LSD1 will outperform LSD1 inhibitors which do not have this dual activity.

In a variation on the procedure above, mice treated with CAR-T alone may be administered LSD1 inhibitor at one or more time points after the CAR-T infusion. For example, when T cells begin to exhibit signs of exhaustion or the antitumor effect begins to wane (i.e., when progression accelerates), LSD1 inhibitor may be administered. It is expected that such subsequent treatment with LDS1i will restore early memory T-cell phenotypes and functions, and that treatment with bomedemstat or another LDS1 inhibitor which inhibits both demethylase activity and the ability of Growth Factor Independent 1 and/or Growth Factor Independent 1b (GFI1 and/or GFI1b and Blimp1) to bind to LSD1 will outperform LSD1 inhibitors which do not have this dual activity.

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A method of treatment of disease comprising administering:
a therapeutic composition comprising a population of immune effector cells prepared for adoptive transfer, which bind to an antigen on the surface of a target cell; and
a lysine-specific demethylase 1 inhibitor;
wherein the lysine-specific demethylase 1 inhibitor is a compound of any of Formulas I-V:
Formula I: 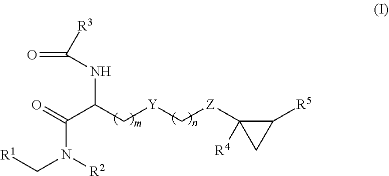
or a salt thereof, wherein:
Y is chosen from a bond, NR^{4a}, O, C(O)NH, NHC(O), S, SO₂, and CH₂;
Z is chosen from a bond, NR^{4b}, O, C(O)NH, NHC(O), S, SO₂, and CH₂;
m is an integer from 0 to 5;
n is an integer from 0 to 3;
R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;
R⁴, R^{4a}, and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;
with the proviso that when Y═CH₂, R⁴═H, and Z═R^{4b}, then m+n≠3;
Formula II: 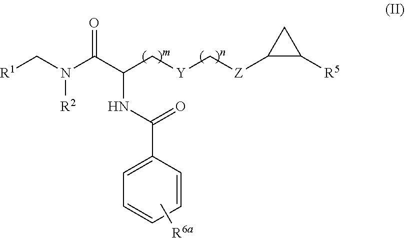
or a salt, polymorph, or solvate thereof, wherein:
Y is chosen from a bond, NR^{4a}, O, C(O)NH, NHC(O), S, SO₂, CHOH, and CH₂;
Z is chosen from a bond, NR^{4b}, O, C(O)NH, NHC(O), S, SO₂, and CH₂;
m is an integer from 0 to 5;
n is an integer from 0 to 3;
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4a} and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
R^{6a} is chosen from heteroaryl, cyano, and S(O)₂N(CH₃)₂;
each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;
with the proviso that when Y═CH₂ and Z═R^{4b}, then m+n≠3;
Formula IIIa or IIIb: 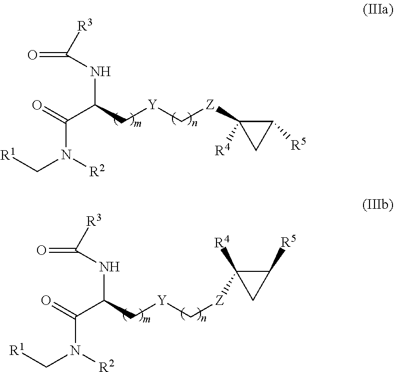
or a salt thereof, wherein:
Y is chosen from a bond, NR^{4a}, O, C(O)NH, NHC(O), S, SO₂, and CH₂;
Z is chosen from a bond, NR^{4b}, O, C(O)NH, NHC(O), S, SO₂, and CH₂;
m is an integer from 0 to 5;
n is an integer from 0 to 3;
R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;
R⁴, R^{4a} and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;
with the proviso that when Y═CH₂ and Z═R^{4b}, then m+n≠3
Formula IV: 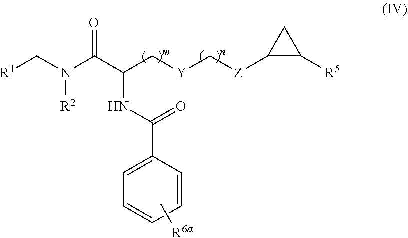
or a salt, polymorph, or solvate thereof, wherein:
Y is chosen from a bond, NR^{4a}, O, C(O)NH, NHC(O), S, SO₂, CHOH, and CH₂;
Z is chosen from a bond, NR^{4b}, O, C(O)NH, NHC(O), S, SO₂, and CH₂;
m is an integer from 0 to 5;
n is an integer from 0 to 3;
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4a} and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
R^{6a} is chosen from heteroaryl, cyano, and S(O)₂N(CH₃)₂;
each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;
with the proviso that when Y═CH₂ and Z═R^{4b}, then m+n≠3;
Formula V: 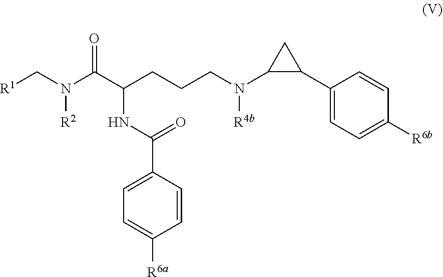
or a salt, polymorph, or solvate thereof, wherein:
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4b} is chosen from hydrogen, alkyl, alkenyl, alkynyl, and cycloalkyl;
R^{6a} is chosen from heteroaryl, cyano, and S(O)₂N(CH₃)₂;
each R⁶ and R^{6b} is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or
R⁷ and R⁸ may be taken together to form a nitrogen-containing heterocycloalkyl or heteroaryl ring, which may be optionally substituted with lower alkyl.

2. The method of claim 1, wherein the lysine-specific demethylase 1 inhibitor is administered concurrently with the therapeutic composition comprising a population of immune effector cells prepared for adoptive transfer.

3. The method of claim 1, wherein the lysine-specific demethylase 1 inhibitor is administered after the therapeutic composition comprising a population of immune effector cells prepared for adoptive transfer.

4. The method of claim 1, wherein the lysine-specific demethylase 1 inhibitor is administered before the therapeutic composition comprising a population of immune effector cells prepared for adoptive transfer.

5. The method of claim 1, wherein the lysine-specific demethylase 1 inhibitor is administered after adoptively transferred immune cell exhaustion is observed.

6. The method of claim 1, wherein the disease is cancer, and the target cell is a cancer cell.

7. The method of claim 1, wherein a memory phenotype of the population of immune effector cells prepared for adoptive transfer is enhanced by treating the immune effector cells with the lysine-specific demethylase 1 inhibitor.

8. The method of claim 1, wherein transition of T cells prepared for adoptive transfer from a memory phenotype to differentiated phenotype is inhibited by treating the immune effector cells with the lysine-specific demethylase 1 inhibitor.

9. The method of claim 1, wherein exhaustion is reversed or inhibited in the population of immune effector cells prepared for adoptive transfer by treating the immune effector cells with the lysine-specific demethylase 1 inhibitor.

10. The method of claim 1, wherein the lysine-specific demethylase 1 inhibitor is administered after adoptively transferred immune cell exhaustion is observed.

11. The method of claim 1, wherein the lysine-specific demethylase 1 inhibitor inhibits both demethylase activity and the ability of Growth Factor Independent 1 and/or Growth Factor Independent 1b (GFI1 and/or GFI1b and Blimp1) to bind to lysine-specific demethylase 1.

12. (canceled)

13. The method of claim 1, wherein the lysine-specific demethylase 1 inhibitor is bomedemstat.

14-16. (canceled)

17. The method of claim 1, wherein the immune effector cells are chosen from T cells and natural killer cells.

18. The method of claim 17, wherein the immune effector cells express at least one chimeric antigen receptor (CAR).

19. The method of claim 18, wherein the immune effector cells are natural killer cells.

20. The method of claim 18, wherein the immune effector cells are T cells.

21-43. (canceled)

44. The method of claim 1, wherein the method is performed in a human subject with cancer.

45. The method of claim 1, wherein the cancer is a hematologic malignancy.

46. The method of claim 45, wherein the hematologic malignancy is a myeloproliferative neoplasm.

47. The method as recited in claim 46, wherein the myeloproliferative neoplasm is myelofibrosis selected from primary myelofibrosis and post polycythemia vera/essential thrombocythemia myelofibrosis.

48. The method as recited in claim 47, wherein the myeloproliferative neoplasm is post polycythemia vera/essential thrombocythemia myelofibrosis (MF).

49. The method as recited in claim 46, wherein the myeloproliferative neoplasm is polycythemia vera.

50. The method as recited in claim 46, wherein the myeloproliferative neoplasm is essential thrombocythemia.

51. The method of claim 45, wherein the hematologic malignancy is acute myeloid leukemia.

52. The method of claim 45, wherein the hematologic malignancy is chronic myeloid leukemia.

53. The method of claim 45, wherein the hematologic malignancy is multiple myeloma.

54. The method of claim 45, wherein the hematologic malignancy is a T-cell malignancy.

55-62. (canceled)
